(19) United States
(12) Patent Application Publication
(10) Pub. No.: US 2017/0217931 A1
(43) Pub. Date: Aug. 3, 2017

(54) COMPOUNDS FOR USE IN ANTHELMINTHIC TREATMENT

(71) Applicant: Bayer Animal Health GMBH, Leverkusen (DE)

(72) Inventors: Claudia WELZ, Düsseldorf (DE); Adeline KÖHLER, Langenfeld (DE); Kirsten BÖRNGEN, Köln (DE); Ulrich GÖRGENS, Ratingen (DE); Hans-Georg SCHWARZ, Dorsten (DE)

(73) Assignee: Bayer Animal Health GMBH, Leverkusen (DE)

(21) Appl. No.: 15/328,854

(22) PCT Filed: Jul. 22, 2015
(86) PCT No.: PCT/EP2015/066726

§ 371 Date: Jan. 24, 2017

(30) Foreign Application Priority Data
Jul. 25, 2014 (EP) 14178621.0
May 29, 2015 (EP) 15169965.9

(51) Int. Cl. C07D 401/14; C07D 401/04; C07D 405/04

(52) U.S. Cl. CPC C07D 401/14; C07D 405/04; C07D 401/04

(57) ABSTRACT

Disclosed are compounds of formula (I) which possess anthelminthic properties wherein the structural elements have the meaning as indicated in the description. Further disclosed are such compounds for the control, treatment and/or prevention of infections with helminths in animals and humans.

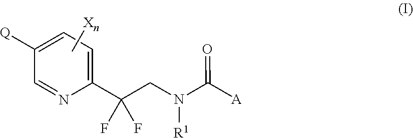

The present invention relates to certain pyridyl carboxamide derivatives. Further, the present invention relates to the use of certain pyridyl carboxamide derivatives for the control, treatment and/or prevention of infections with helminths in animals and humans, formulations containing such compounds and methods for the control, treatment and/or prevention of infections with helminths in animals and humans.

The occurrence of resistances against all commercial anthelmintics seems to be a growing problem in the area of veterinary medicine. Therefore, endoparasiticides with new molecular modes of action are urgently desired. The new active ingredients should perform with excellent efficacy against a broad spectrum of helminths, like nematodes, preferably without any adverse toxic effects to the treated organism. Endoparasiticides are pharmaceuticals for combat or suppression of endoparasites in animals or humans.

The use of certain N-2-(pyridyl)ethyl-carboxamide derivatives for controlling nematodes is described in WO 2007/108483 A1 and EP 2 132 987 A1.

The use of certain carboxamides as parasiticides is described in WO 2012/118139 A1, WO 2013/0676230 A1, WO 2014/034750 A1 and WO 2014/034751 A1.

Furthermore, certain carboxamides are described as pesticides in WO 2013/064518 A1, WO 2013/064519 A1, WO 2013/064520 A1, WO 2013/064521 A1, WO 2014/004064 A1 or as nematicides in WO 2013/064460 A1 and WO 2013/064461 A1.

Also, certain carboxamides, herein mentioned as Examples 1, 2 and 3, are described in the European patent application with the application No. EP 13181692.8.

It is an object of the present invention to provide compounds which can be used as endoparasiticides in the medical, especially veterinary, field with a satisfactory or improved anthelmintic activity against a broad spectrum of helminths, like nematodes, particularly at relatively low dosages, for the control, treatment and/or prevention of infections with helminths in animals and humans, preferably without any adverse toxic effects to the treated organism.

The present invention relates to compounds of formula (I)

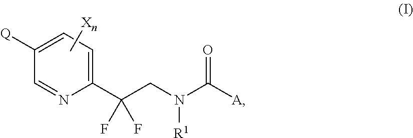

wherein
R¹ is selected from the group consisting of hydrogen, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl, —S(O)₂—C₁-C₄-alkyl, and —S(O)₂—C₁-C₄-halogenoalkyl having 1 to 5 halogen atoms,
n is 0, 1, 2 or 3,
each X is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino,
Q represents an aromatic 5-membered heterocyclic ring containing one to four heterotaoms chosen from N, S and O and bearing the substituent(s) Yₘ, with
   m is 0, 1, 2, 3 or 4, limited by the number of available positions in Q to which a substituent Y can be connected, and
   each Y is independently selected from the group consisting of hydrogen, oxo, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, —CH₂—S—C₁-C₈-alkyl, —CH₂—S(O)—C₁-C₈-alkyl, —CH₂—S(O)₂—C₁-C₈-alkyl, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino, and
A represents a phenyl group of the formula (A1)

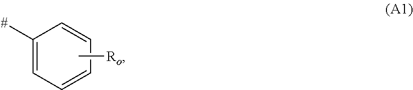

in which
# depicts the bond which connects A to the rest of the molecule,
o is 0, 1, 2, 3, 4 or 5, and
each R is independently selected from the group consisting of halogen, nitro, —OH, NH₂, SH, SF₅, CHO, OCHO, NHCHO, COOH, cyano, C₁-C₈-alkyl, C₁-C₈-halogenoalkyl having 1 to 9 halogen atoms, C₂-C₈-alkenyl, C₂-C₈-alkynyl, C₃-C₆-cycloalkyl, —S—C₁-C₈-alkyl, —S—C₁-C₈-halogenoalkyl having 1 to 5 halogen atoms, C₁-C₈-alkoxy, C₁-C₈-halogenoalkoxy having 1 to 5 halogen atoms, C₁-C₈-alkoxy-C₂-C₈-alkenyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, C₁-C₈-alkylsulfonamide, —NH(C₁-C₈-alkyl), N(C₁-C₈-alkyl)₂, phenyl (optionally substituted by C₁-C₆-alkoxy) and phenoxy, or two R bonded to adjacent carbon atoms together represent —O(CH₂)ₚO—, wherein p represents 1 or 2, or
A represents a heterocycle of the formula (Het-1)

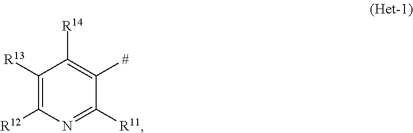

in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₈-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and —S-phenyl (optionally substituted by halogen or C₁-C₄-alkyl), and
R¹², R¹³ and R¹⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, or
A represents a heterocycle of the formula (Het-2)

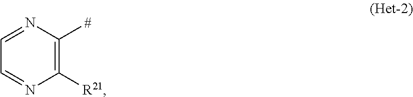

in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms,
or a pharmaceutically acceptable salt, N-oxide, metal complex or metalloid complex thereof,
with the proviso that if
   A is

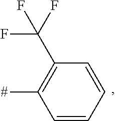

in which
# depicts the bond which connects A to the rest of the molecule,
R¹ is hydrogen,
X is chlorine at position 3 of the pyridine ring where it is connected to, and
n is 1, then
Q is not one of the following

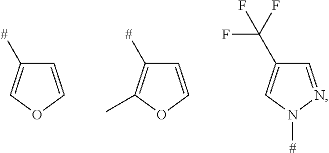

in which
# depicts the bond which connects Q to the rest of the molecule.

The present invention relates further to compounds of formula (I)

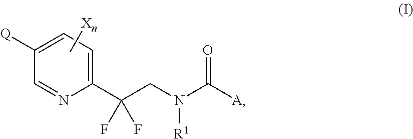

wherein
R¹ is selected from the group consisting of hydrogen, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl, —S(O)₂—C₁-C₄-alkyl, and —S(O)₂—C₁-C₄-halogenoalkyl having 1 to 5 halogen atoms,
n is 0, 1, 2 or 3,
each X is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino,
Q represents an aromatic 5-membered heterocyclic ring containing one to four heterotaoms chosen from N, S and O and bearing the substituent(s) Yₘ, with
   m is 0, 1, 2, 3 or 4, limited by the number of available positions in Q to which a substituent Y can be connected, and
   each Y is independently selected from the group consisting of hydrogen, oxo, halogen, nitro, cyano, hydroxy, amino, —SH, —SF₅, —CHO, —OCHO, —NHCHO, —COOH, —CONH₂, —CONH(OH),
      —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, —CH₂—S—C₁-C₈-alkyl, —CH₂—S(O)—C₁-C₈-alkyl, —CH₂—S(O)₂—C₁-C₈-alkyl, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino, and
A represents a phenyl group of the formula (A1)

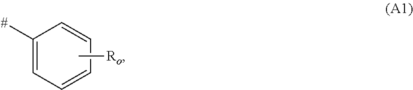

in which
# depicts the bond which connects A to the rest of the molecule,
o is 0, 1, 2, 3, 4 or 5, and
each R is independently selected from the group consisting of halogen, nitro, —OH, NH₂, SH, SF₅, CHO, OCHO, NHCHO, COOH, cyano, C₁-C₈-alkyl, C₁-C₈-halogenoalkyl having 1 to 9 halogen atoms, C₂-C₈-alkenyl, C₂-C₈-alkynyl, C₃-C₆-cycloalkyl, —S—C₁-C₈-alkyl, —S—C₁-C₈-halogenoalkyl having 1 to 5 halogen atoms, C₁-C₈-alkoxy, C₁-C₈-halogenoalkoxy having 1 to 5 halogen atoms, C₁-C₈-alkoxy-C₂-C₈-alkenyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, C₁-C₈-alkylsulfonamide, —NH(C₁-C₈-alkyl), N(C₁-C₈-alkyl)₂, phenyl (optionally substituted by C₁-C₆-alkoxy) and phenoxy, or two R bonded to adjacent carbon atoms together represent —O(CH₂)ₚO—, wherein p represents 1 or 2, or
A represents a heterocycle of the formula (Het-1)

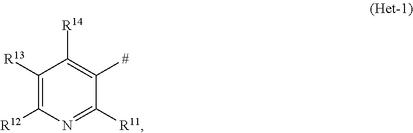

in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₈-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and —S-phenyl (optionally substituted by halogen or C₁-C₄-alkyl), and
R¹², R¹³ and R¹⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, or
A represents a heterocycle of the formula (Het-2)

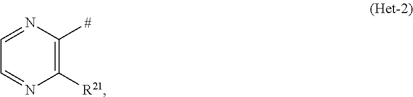

in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms,
or a pharmaceutically acceptable salt, N-oxide, metal complex or metalloid complex thereof,
for use in the control, treatment and/or prevention of infections with helminths in animals and humans.

In formulae (Al), (Het-1) and (Het-2) # depicts the bond which connects A to the C(O)NR¹-moiety in the compounds of formula (I) or formula (I-1). In the context of the formulae describing the residue Q, # depicts the bond which connects Q to the pyridine-moiety of formula (I) or formula (I-1). In general, in the present application # depicts the connecting bond of the structural element, unless otherwise indicated.

Any of the compounds according to the invention can exist in one or more optical or chiral isomer forms depending on the number of asymmetric centres in the compound. The invention thus relates equally to all the optical isomers and to their racemic or scalemic mixtures (the term “scalemic” denotes a mixture of enantiomers in different proportions), and to the mixtures of all the possible stereoisomers, in all proportions. The diastereoisomers and/or the optical isomers can be separated according to the methods which are known per se by the man ordinary skilled in the art.

Compounds of the present invention can also exist in one or more geometric isomer forms depending on the number of double bonds in the compound, especially all syn/anti (or cis/trans) isomers and to all possible syn/anti (or cis/trans) mixtures. The invention thus relates equally to all geometric isomers and to all possible mixtures, in all proportions. The geometric isomers can be separated according to general methods, which are known per se by the man ordinary skilled in the art.

Compounds of formula (I) may be found in its tautomeric form resulting from the shift of the proton of a hydroxy, sulfanyl or amino group. Such tautomeric forms of such compounds are also part of the present invention. More generally speaking, all tautomeric forms of compounds of formula (I), as well as the tautomeric forms of the compounds which can optionally be used as intermediates in the preparation processes and which will be defined in the description of these processes, are also part of the present invention.

Further, this invention is directed to pharmaceutical compositions comprising a compound of the invention. Furthermore, this invention is directed to pharmaceutical compositions comprising a compound of the invention for use in the control, treatment and/or prevention of infections with helminths in animals and humans. This invention also provides a composition comprising a compound of formula (I), or an N-oxide, or a pharmaceutically acceptable salt thereof, and at least one pharmaceutically acceptable excepient. In one embodiment, this invention provides such a composition which further comprises at least one additional active ingredient, preferably a mixing partner as described below.

Further, this invention is directed to the use of compounds and/or compositions of the invention for the control, treatment and/or prevention of infections with helminths in animals and humans. This invention provides also a method for control, treatment and/or prevention of infections with helminths in animals and humans comprising administering a biologically effective amount of a compound of formula (I), or an N-oxide, or a pharmaceutically acceptable salt thereof, or a composition described herein, to an animal or human in need. This invention also relates to such method wherein a composition comprising a biologically effective amount of a compound of formula (I), an N-oxide or a pharmaceutically acceptable salt thereof, and at least one pharmaceutically acceptable excipient, is administered to an animal or human in need, said composition optionally further comprising a biologically effective amount of at least one additional active ingredient, preferably a mixing partner as described below. According to the present invention, the described uses and methods are applicable in the context of the control, treatment and/or prevention of infections with helminths in animal and humans. If at any point any such use or method is only mentioned with regard to animals, this shall in general, and unless specifically indicated otherwise, refer to the use/method with regard to animals and humans and shall not be understood as a limitation. However, the uses and methods according to the present invention are in one preferred embodiment directed to the control, treatment and/or prevention of infections with helminths in non-human animals (only). In one embodiment, the methods according to the invention do not comprise methods for treatment of the human body by surgery or therapy and diagnostic methods practised on the human body.

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising”, it should be readily understood that (unless otherwise stated) the description should be interpreted to also describe such an invention using the terms “consisting essentially of” or “consisting of”.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

In the above recitations, the term “alkyl”, used either alone or in compound words such as “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂.

“Cycloalkyl” includes, for example, cyclopropyl, cyclobutyl, cyclopentyl and cyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. “Cycloalkenyl” includes groups such as cyclopentenyl and cyclohexenyl as well as groups with more than 10 one double bond such as 1,3- and 1,4-cyclohexadienyl. The term “cycloalkylcycloalkyl” denotes cycloalkyl substitution on another cycloalkyl ring, wherein each cycloalkyl ring independently has from 3 to 7 carbon atom ring members. Examples of cycloalkylcycloalkyl include cyclopropylcyclopropyl (such as 1,1′-bicyclopropyl-1-yl, 1,1′-bicyclopropyl-2-yl), cyclohexylcyclopentyl (such as 4-cyclopentylcyclohexyl) and cyclohexylcyclohexyl (such as 1,1′-bicyclohexyl-1-yl), and the different cis- and trans-cycloalkylcycloalkyl isomers, (such as (1R,2S)-1,1′-bicyclopropyl-2-yl and (1R,2R)-1,1′-bicyclopropyl-2-yl).

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “haloalkoxy”, “haloalkenyl”, “haloalkynyl”, and the like, are defined analogously to the term “haloalkyl”. Examples of “haloalkoxy” include CF₃O, CCl₃CH₂O, HCF₂CH₂CH₂O and CF₃CH₂O. Examples of “haloalkenyl” include (Cl)₂C═CHCH₂ and CF₃CH₂CH═CHCH₂. Examples of “haloalkynyl” include HC≡CCHC1, CF₃C≡C, CCl₃C≡C and FCH₂C≡CCH₂.

The chemical abbreviation C(O) as used herein represents a carbonyl moiety. For example, C(O)CH₃ represents an acetyl group. The chemical abbreviations CO₂ and C(O)O as used herein represent an ester moiety. For example, CO₂Me and C(O)OMe represent a methyl ester. CHO represents an aldehyde moiety.

“OCN” means —O—C≡N, and “SCN” means —S—C≡N.

The total number of carbon atoms in a substituent group is indicated by the “Ci-Cj” prefix where i and j are numbers from 1 to 14. C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

When a compound is substituted with a substituent bearing a subscript that indicates the number of said substituents can exceed 1, said substituents (when they exceed 1) are independently selected from the group of defined substituents, e.g. n=0, 1, 2, 3 or 4. When a group contains a substituent which can be hydrogen, for example R² or R³, then when this substituent is taken as hydrogen, it is recognized that this is equivalent to said group being unsubstituted.

Unless otherwise indicated, a “ring” or “ring system” as a component of formula (I) is carbocyclic or heterocyclic. The term “ring system” denotes two or more fused rings. The term “heterocyclic ring” denotes a ring in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. The term “heterocyclic ring system” denotes a ring system in which at least one ring of the ring system is a heterocyclic ring. Unless otherwise indicated, heterocyclic rings and ring systems can be attached through any available carbon or nitrogen by replacement of a hydrogen on said carbon or nitrogen.

As used herein, the following definitions shall apply unless otherwise indicated. The term “optionally substituted” is used interchangeably with the phrase “substituted or unsubstituted” or with the term “(un)substituted”. The expression “optionally substituted with 1 to 4 substituents” means that no substituent is present (i.e. unsubstituted) or that 1, 2, 3 or 4 substituents are present (limited by the number of available bonding positions). Unless otherwise indicated, an optionally substituted group may have a substituent at each substitutable position of the group, and each substitution is independent of the other.

The following embodiments that relate to formula (I) shall be understood as referring to the compounds according to the present invention per se or to the compounds for use in the control, treatment and/or prevention of infections with helminths in animals and humans according to the present invention, or to both.

In one embodiment, the present invention provides compounds of formula (I), wherein
Q represents a 5-membered ring selected from the group consisting of Q-1 to Q-47:

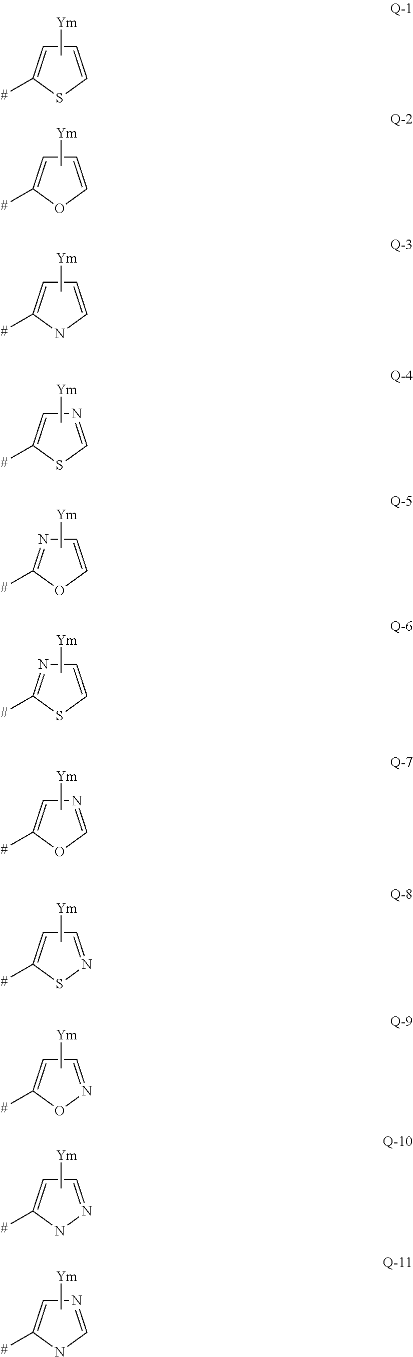

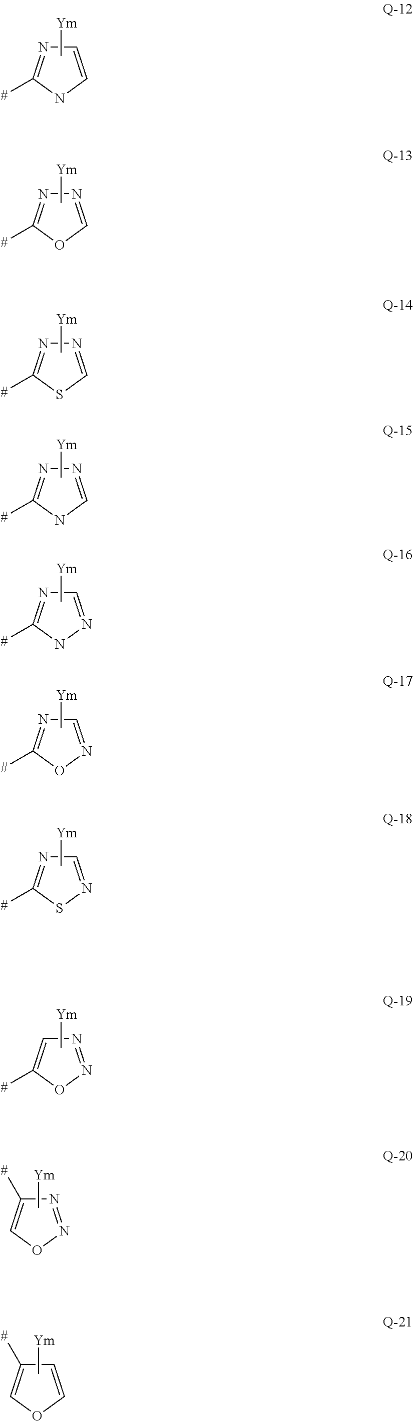

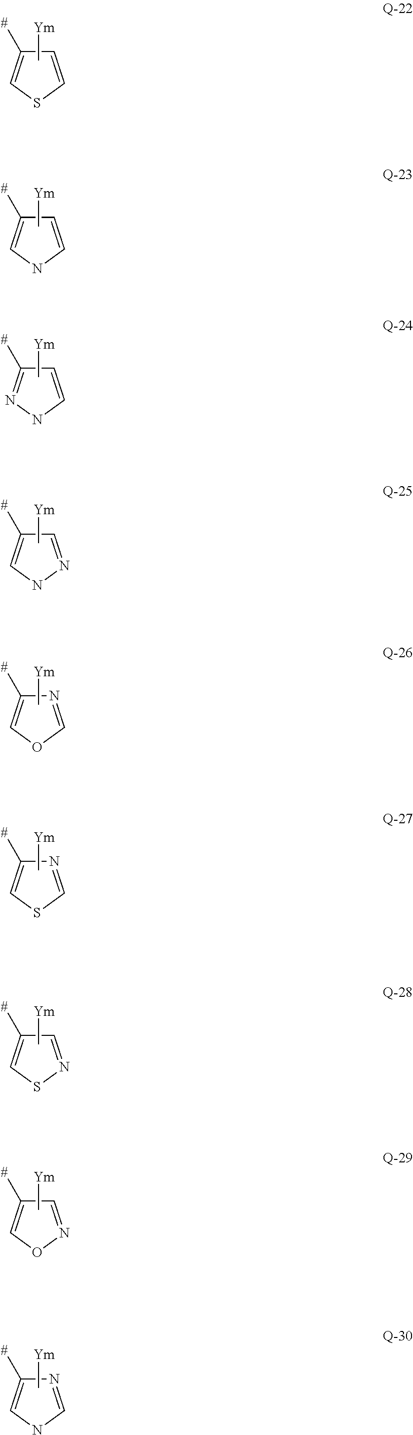

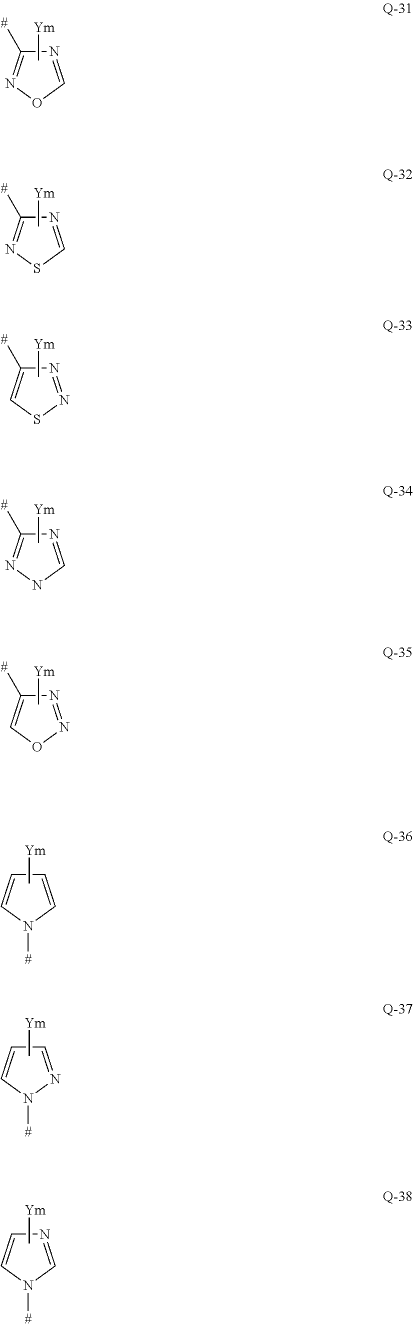

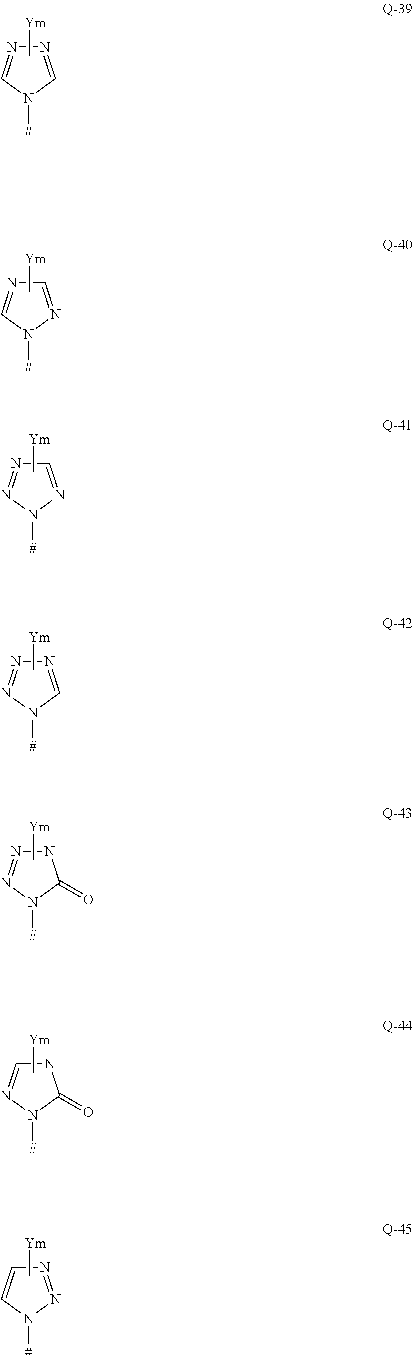

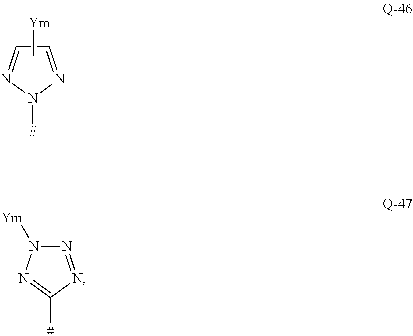

in which
# depicts the bond which connects Q to the rest of the molecule,
with m and Y having the meaning as described before, and
R¹, n, X, and A have a meaning as described herein.

According to a further embodiment, the present invention provides compounds according to formula (I), wherein
Q represents a 5-membered ring selected from the group consisting of Q-1 to Q-47, with
   m is 0, 1 or 2, limited by the number of available positions in Q to which a substituent Y can be connected, and
   each Y is independently selected from the group consisting of hydrogen, halogen, nitro, cyano, C₁-C₄-alkyl, C₁-C₄-halogenoalkyl having 1 to 5 halogen atoms, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, —CH₂—S—C₁-C₄-alkyl, —CH₂—S(O)—C₁-C₄-alkyl, —CH₂—S(O)₂—C₁-C₄-alkyl, (C₁-C₄-alkoxyimino)-C₁-C₄-alkyl, (C₂-C₆-alkenyloxyimino)-C₁-C₄-alkyl, (C₃-C₆-alkynyloxyimino)-C₁-C₄-alkyl, (benzyloxyimino)-C₁-C₆-alkyl, benzyloxy, —S-benzyl, benzylamino, phenoxy, —S-phenyl and phenylamino, and
R¹, n, X, and A have a meaning as described herein.

According to a still further embodiment, the present invention provides compounds according to formula (I), wherein
Q represents a 5-membered ring selected from the group consisting of Q-1 to Q-47, with
   m is 0, 1 or 2, limited by the number of available positions in Q to which a substituent Y can be connected, and
   each Y is independently selected from the group consisting of hydrogen, halogen, C₁-C₄-alkyl and C₁-C₄-halogenoalkyl having 1 to 5 halogen atoms, and
R¹, n, X, and A have a meaning as described herein.

According to a still further embodiment, the present invention provides compounds according to formula (I), wherein
Q represents a 5-membered ring selected from the group consisting of Q-1, Q-2, Q-3, Q-4, Q-5, Q-6, Q-7, Q-8, Q-9, Q-10, Q-11, Q-12, Q-13, Q-14, Q-15, Q-16, Q-18, Q-21, Q-22, Q-23, Q-24, Q-25, Q-26, Q-27, Q-28, Q-29, Q-30, Q-31, Q-32, Q-33, Q-34, Q-36, Q-37, Q-38, Q-39, Q-40, Q-41 and Q-44,
   with m and Y having a meaning as described before, and
R¹, n, X, and A have a meaning as described herein.

According to a still further embodiment, the present invention provides compounds according to formula (I), wherein
Q represents a 5-membered ring selected from the group consisting of Q-1, Q-4, Q-6, Q-10, Q-21, Q-23, Q-24, Q-25, Q-27, Q-37, Q-41 and Q-44,
   with m and Y having a meaning as described before, and
R¹, n, X, and A have a meaning as described herein.

According to a still further embodiment, the present invention provides compounds according to formula (I), wherein
Q represents a 5-membered ring selected from the group consisting of Q-21, Q-23, Q-25, Q-37 and Q-44,
   with m and Y having a meaning as described before, and
R¹, n, X, and A have a meaning as described herein.

In another embodiment, the present invention provides compounds according to formula (I), wherein
n is 1 or 2, limited by the number of available positions in the ring to which a substituent X can be connected,
each X is independently selected from the group consisting of hydrogen, halogen, nitro, cyano, C₁-C₄-alkyl, C₁-C₄-halogenoalkyl having 1 to 5 halogen atoms, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, (C₁-C₄-alkoxyimino)-C₁-C₄-alkyl, (C₂-C₆-alkenyloxyimino)-C₁-C₄-alkyl, (C₃-C₆-alkynyloxyimino)-C₁-C₄-alkyl, (benzyloxyimino)-C₁-C₆-alkyl, benzyloxy, —S-benzyl, benzylamino, phenoxy, —S-phenyl and phenylamino, and
R¹, Q and A have a meaning as described herein.

According to a further embodiment, the present invention provides compounds according to formula (I), wherein
n is 1 or 2, limited by the number of available positions in the ring to which a substituent X can be connected,
each X is independently selected from the group consisting of halogen and C₁-C₄-halogenoalkyl having 1 to 5 halogen atoms, and
R¹, Q and A have a meaning as described herein.

According to a still further embodiment, the present invention provides compounds according to formula (I), wherein
n is 1 or 2, limited by the number of available positions in the ring to which a substituent X can be connected,
each X is independently selected from the group consisting of halogen and CF₃, and
R¹, Q and A have a meaning as described herein.

According to a still further embodiment, the present invention provides compounds according to formula (I), wherein
n is 1 or 2, limited by the number of available positions in the ring to which a substituent X can be connected,
X is Cl, and
R¹, Q and A have a meaning as described herein.

According to a still further embodiment, the present invention provides compounds according to formula (I), wherein
n is 1, and
R¹, X, Q and A have a meaning as described herein.

In another embodiment, the present invention provides compounds according to formula (I), wherein
A represents a phenyl group of formula (A1)

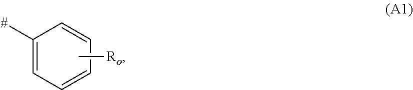

in which
# depicts the bond which connects A to the rest of the molecule,
o is 0, 1 or 2, and
each R is independently selected from the group consisting of halogen, nitro, —OH, CHO, OCHO, NHCHO, cyano, C₁-C₄-alkyl, C₁-C₄-halogenoalkyl having 1 to 5 halogen atoms, C₂-C₄-alkenyl, C₂-C₄-alkynyl, C₃-C₆-cycloalkyl, —S—C₁-C₄-alkyl, —S—C₁-C₄-halogenoalkyl having 1 to 5 halogen atoms, C₁-C₄-alkoxy, C₁-C₄-halogenoalkoxy having 1 to 5 halogen atoms, C₁-C₄-alkoxy-C₂-C₄-alkenyl, C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylsulfonamide, —NH(C₁-C₄-alkyl), N(C₁-C₄-alkyl)₂, phenyl (optionally substituted by C₁-C₄-alkoxy) and phenoxy, or two R bonded to adjacent carbon atoms together represent —O(CH₂)ₚO—, wherein p represents 1 or 2, or
A represents a heterocycle of the formula (Het-1)

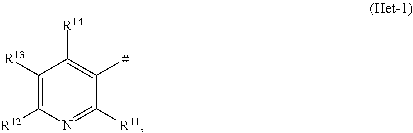

in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₈-alkyl, —S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and —S-phenyl (optionally substituted by halogen or C₁-C₄-alkyl), and
R¹², R¹³ and R¹⁴, which may the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, or
A represents a heterocycle of the formula (Het-2)

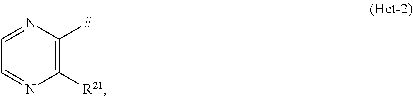

in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms, and
R¹, n, X and Q have a meaning as described herein.

According to a further embodiment, the present invention provides compounds according to formula (I), wherein
A represents a phenyl group of formula (A1)

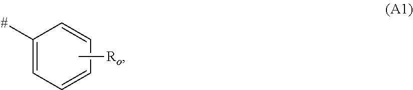

in which
# depicts the bond which connects A to the rest of the molecule,
o is 0, 1 or 2, and
each R is independently selected from the group consisting of halogen, nitro, C₁-C₄-halogenoalkyl having 1 to 5 halogen atoms, C₁-C₄-halogenoalkoxy having 1 to 5 halogen atoms, or
A represents a heterocycle of the formula (Het-1)

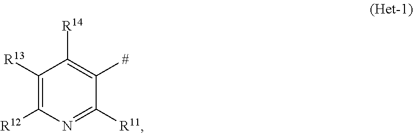

in which
# depicts the bond which connects A to the rest of the molecule,
R¹¹ is C₁-C₄-halogenoalkyl having 1 to 5 halogen atoms, and
R¹², R¹³ and R¹⁴ are hydrogen, or
A represents a heterocycle of the formula (Het-2)

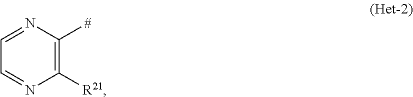

in which
# depicts the bond which connects A to the rest of the molecule, and
R²¹ is C₁-C₄-halogenoalkyl having 1 to 5 halogen atoms, and
R¹, n, X and Q have a meaning as described herein.

According to a still further embodiment, the present invention provides compounds according to formula (I), wherein
A represents a phenyl group of formula (A1)

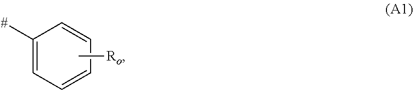

in which
# depicts the bond which connects A to the rest of the molecule,
o is 1 or 2, and
each R is independently selected from the group consisting of halogen, nitro, —CF₃, —CHF₂, —OCF₃, or
A represents a heterocycle of the formula (Het-1)

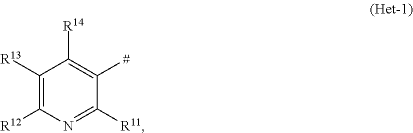

in which
# depicts the bond which connects A to the rest of the molecule,
R¹¹ is CF₃, and
R¹², R¹³ and R¹⁴ are hydrogen, or
A represents a heterocycle of the formula (Het-2)

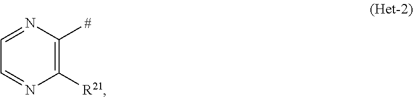

in which
# depicts the bond which connects A to the rest of the molecule, and
R²¹ is CF₃, and
R¹, n, X and Q have a meaning as described herein.

According to a still further embodiment, the present invention provides compounds according to formula (I), wherein
A is selected from the group consisting of

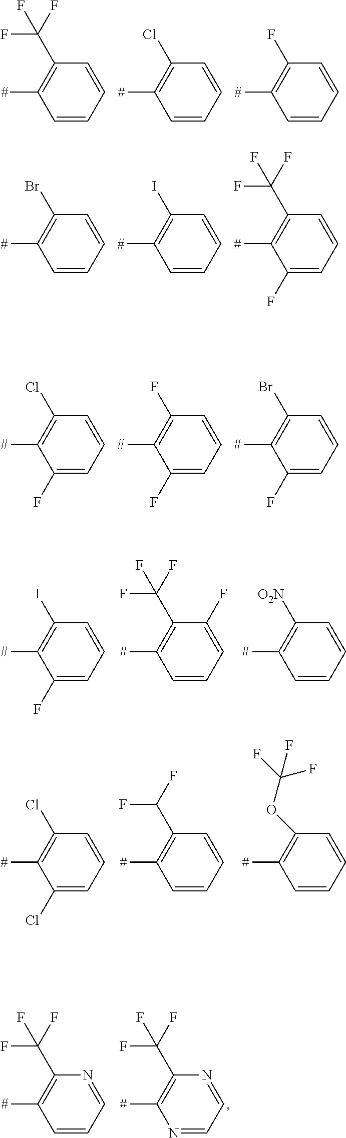

in which
# depicts the bond which connects A to the rest of the molecule, and
R¹, n, X and Q have a meaning as described herein.

According to a still further embodiment, the present invention provides compounds according to formula (I), wherein
A is selected from the group consisting of

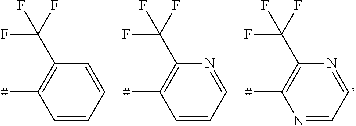

in which
# depicts the bond which connects A to the rest of the molecule, and
R¹, n, X and Q have a meaning as described herein.

According to a still further embodiment, the present invention provides compounds according to formula (I), wherein
A is

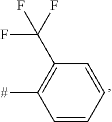

in which
# depicts the bond which connects A to the rest of the molecule, and
R¹, n, X and Q have a meaning as described herein.

In another embodiment, the present invention provides compounds according to formula (I),
wherein A, R¹, n, X and Q have a meaning as described herein,
   with the proviso that if
A is

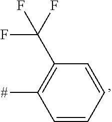

in which
# depicts the bond which connects A to the rest of the molecule,
R¹ is hydrogen,
X is chlorine at position 3 of the pyridine ring where it is connected to, and
n is 1, then
Q is not Q-21 or
Q is not Q-37 or
Q is not Q-21 and Q-37.

Aside from the individual embodiments, any possible combination of the afore-mentioned individual embodiments provides compounds according to formula (I) within the scope of the present invention.

These combinations lead to additional particular embodiments within the scope of the present invention, some of which are illustrated by the following specific embodiments by way of example.

In still another embodiment, the present invention provides compounds according to formula (I), wherein
n is 1 or 2, limited by the number of available positions in the ring to which a substituent X can be connected,
each X is independently selected from the group consisting of hydrogen, halogen, nitro, cyano, C₁-C₄-alkyl, C₁-C₄-halogenoalkyl having 1 to 5 halogen atoms, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, (C₁-C₄-alkoxyimino)-C₁-C₄-alkyl, (C₂-C₆-alkenyloxyimino)-C₁-C₄-alkyl, (C₃-C₆-alkynyloxyimino)-C₁-C₄-alkyl, (benzyloxyimino)-C₁-C₆-alkyl, benzyloxy, —S-benzyl, benzylamino, phenoxy, —S-phenyl and phenylamino,
Q represents a 5-membered ring selected from the group consisting of Q-1, Q-2, Q-3, Q-4, Q-5, Q-6, Q-7, Q-8, Q-9, Q-10, Q-11, Q-12, Q-13, Q-14, Q-15, Q-16, Q-17, Q-18, Q-19, Q-20, Q-21, Q-22, Q-23, Q-24, Q-25, Q-26, Q-27, Q-28, Q-29, Q-30, Q-31, Q-32, Q-33, Q-34, Q-35, Q-36, Q-37, Q-38, Q-39, Q-40, Q-41, Q-42, Q-43, Q-44, Q-45, Q-46 and Q-47, with
   m is 0, 1 or 2, limited by the number of available positions in Q to which a substituent Y can be connected, and
   each Y is independently selected from the group consisting of hydrogen, halogen, nitro, cyano, C₁-C₄-alkyl, C₁-C₄-halogenoalkyl having 1 to 5 halogen atoms, C₂-C₄-alkenyl, C₂-C₄-alkynyl, C₁-C₄-alkylamino, di-(C₁-C₄-alkyl)amino, C₁-C₄-alkoxy, C₁-C₄-halogenoalkoxy having 1 to 5 halogen atoms, C₂-C₄-alkenyloxy, C₂-C₄-halogenoalkenyloxy having 1 to 5 halogen atoms, C₃-C₄-alkynyloxy, C₃-C₄-halogenoalkynyloxy having 1 to 5 halogen atoms, C₃-C₆-cycloalkyl, C₃-C₆-halogenocycloalkyl having 1 to 5 halogen atoms, C₁-C₄-alkylcarbonyl, C₁-C₄-halogenoalkylcarbonyl having 1 to 5 halogen atoms, —CONH(C₁-C₄-alkyl), —CON(C₁-C₄-alkyl)₂, —CONH(OC₁-C₄-alkyl), —CON(OC₁-C₄-alkyl)(C₁-C₄-alkyl), C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, C₁-C₄-alkylcarbonylamino, C₁-C₄-halogenoalkylcarbonylamino having 1 to 5 halogen atoms, —OCONH(C₁-C₄-alkyl), —OCON(C₁-C₄-alkyl)₂, —OCONH(OC₁-C₄-alkyl), —OCO(OC₁-C₄-alkyl), —S—C₁-C₄-alkyl, —S—C₁-C₄-halogenoalkyl having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, —CH₂—S—C₁-C₄-alkyl, —CH₂—S(O)—C₁-C₄-alkyl, —CH₂—S(O)₂—C₁-C₄-alkyl, (C₁-C₄-alkoxyimino)-C₁-C₄-alkyl, (C₂-C₆-alkenyloxyimino)-C₁-C₄-alkyl, (C₃-C₆-alkynyloxyimino)-C₁-C₄-alkyl, (benzyloxyimino)-C₁-C₆-alkyl, benzyloxy, —S-benzyl, benzylamino, phenoxy, —S-phenyl and phenylamino,
R¹ is selected from the group consisting of hydrogen, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl, —S(O)₂—C₁-C₄-alkyl, and —S(O)₂—C₁-C₄-halogenoalkyl having 1 to 5 halogen atoms, and
A represents a phenyl group of formula (A1)

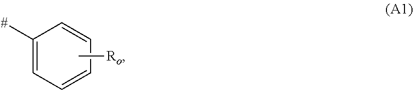

in which
# depicts the bond which connects A to the rest of the molecule,
o is 0, 1 or 2, and
each R is independently selected from the group consisting of halogen, nitro, —OH, CHO, OCHO, NHCHO, cyano, C₁-C₄-alkyl, C₁-C₄-halogenoalkyl having 1 to 5 halogen atoms, C₂-C₄-alkenyl, C₂-C₄-alkynyl, C₃-C₆-cycloalkyl, —S—C₁-C₄-alkyl, —S—C₁-C₄-halogenoalkyl having 1 to 5 halogen atoms, C₁-C₄-alkoxy, C₁-C₄-halogenoalkoxy having 1 to 5 halogen atoms, C₁-C₄-alkoxy-C₂-C₄-alkenyl, C₁-C₄-alkoxycarbonyl, C₁-C₄-halogenoalkoxycarbonyl having 1 to 5 halogen atoms, C₁-C₄-alkylcarbonyloxy, C₁-C₄-halogenoalkylcarbonyloxy having 1 to 5 halogen atoms, —S(O)—C₁-C₄-alkyl, —S(O)—C₁-C₄-halogenoalkyl having 1 to 5 halogen atoms, —S(O)₂—C₁-C₄-alkyl, —S(O)₂—C₁-C₄-halogenoalkyl having 1 to 5 halogen atoms, C₁-C₄-alkylsulfonamide, —NH(C₁-C₄-alkyl), N(C₁-C₄-alkyl)₂, phenyl (optionally substituted by C₁-C₄-alkoxy) and phenoxy, or two R bonded to adjacent carbon atoms together represent —O(CH₂)ₚO—, wherein p represents 1 or 2, or
A represents a heterocycle of the formula (Het-1)

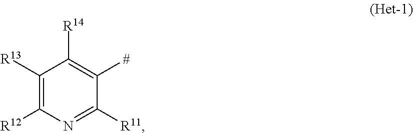

in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₈-alkyl, —S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and —S-phenyl (optionally substituted by halogen or C₁-C₄-alkyl), and
R¹², R¹³ and R¹⁴, which may the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, or
A represents a heterocycle of the formula (Het-2)

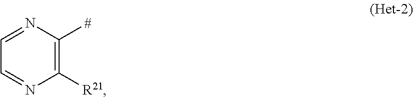

in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms.

In yet another embodiment, the present invention provides compounds according to formula (I), wherein
n is 1,
X is selected from the group consisting of hydrogen, halogen, nitro, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
Q represents an optionally mono- or polysubstituted heteroaromatic ring from the group consisting of Q-1, Q-2, Q-3, Q-4, Q-5, Q-6, Q-7, Q-8, Q-9, Q-10, Q-11, Q-12, Q-13, Q-14, Q-15, Q-16, Q-18, Q-21, Q-22, Q-23, Q-24, Q-25, Q-26, Q-27, Q-28, Q-29, Q-30, Q-31, Q-32, Q-33, Q-34, Q-36, Q-37, Q-38, Q-39, Q-40, Q-41 and Q-44,
   with
   m is 0, 1 or 2, limited by the number of available positions in Q to which a substituent Y can be connected, and
   each Y is independently selected from the group consisting of hydrogen, —CF₃, —CH₂CF₃, methyl, ethyl, fluorine, chlorine, bromine, iodine, cyano, —OCH₃, —OCH₂CH₃, —OCH(CH₃)₂, —OCH₂CF₃, —CH₂—S(O)₂—CH₃,
R¹ is selected from the group consisting of hydrogen, C₁-C₄-alkyl, C₁-C₄-alkoxy-C₁-C₄-alkyl, C₃-C₆-cycloalkyl, C₁-C₄-alkylcarbonyl, C₁-C₄-alkoxycarbonyl, and
A represents a phenyl group of formula (A1)

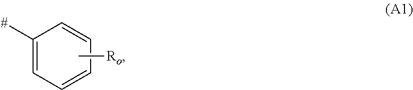

in which
# depicts the bond which connects A to the rest of the molecule,
o is 0, 1 or 2, and
each R is independently selected from the group consisting of halogen, nitro, —OH, cyano, C₁-C₄-alkyl, C₁-C₄-halogenoalkyl having 1 to 5 halogen atoms, C₃-C₆-cycloalkyl, C₁-C₄-alkoxy, C₁C₁-C₄-alkoxycarbonyl, —NH(C₁-C₄-alkyl), phenyl (optionally substituted by C₁-C₄-alkoxy) and phenoxy, or
A represents a heterocycle of the formula (Het-1)

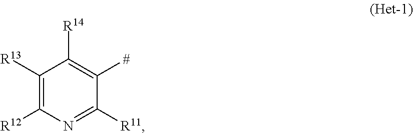

in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₈-alkyl, —S(O)—C₁-C₄-alkyl, —S(O)₂C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and —S-phenyl (optionally substituted by halogen or C₁-C₄-alkyl), and
R¹², R¹³ and R¹⁴, which may the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, or
A represents a heterocycle of the formula (Het-2)

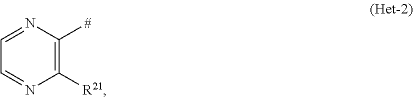

in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms.

In yet another embodiment, the present invention provides compounds according to formula (I), wherein
n is 1,
X is selected from the group consisting of hydrogen, halogen, nitro, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
Q represents a 5-membered ring selected from the group consisting of Q-1, Q-4, Q-6, Q-10, Q-21, Q-23, Q-24, Q-25, Q-27, Q-37, Q-41 and Q-44, with
   m is 0, 1 or 2, limited by the number of available positions in Q to which a substituent Y can be connected, and
   each Y is independently selected from the group consisting of hydrogen, —CF₃, —CH₂CF₃, methyl, ethyl, fluorine, chlorine, bromine, iodine, cyano, —OCH₃, —OCH₂CH₃,
R¹ is hydrogen, and
A represents a phenyl group of formula (A1)

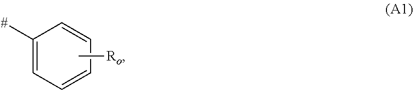

in which
# depicts the bond which connects A to the rest of the molecule,
o is 1 or 2, and
each R is independently selected from the group consisting of halogen, nitro, —OH, cyano, C₁-C₄-alkyl, C₁-C₄-halogenoalkyl having 1 to 5 halogen atoms, C₃-C₆-cycloalkyl, C₁-C₄-alkoxy, or
A represents a heterocycle of the formula (Het-1)

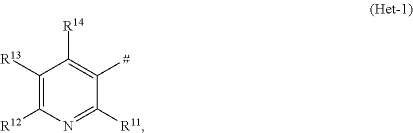

in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, C₁-C₄-halogenoalkoxy having 1 to 5 halogen atoms, and
R¹², R¹³ and R¹⁴, which may the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, or
A represents a heterocycle of the formula (Het-2)

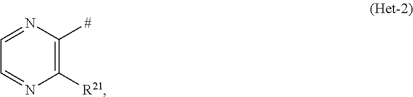

in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms.

In yet another embodiment, the present invention provides compounds according to formula (I), wherein
n is 1,
X is selected from the group consisting of hydrogen, halogen, —CF₃,
Q represents a 5-membered ring selected from the group consisting of Q-21, Q-23, Q-25, Q-37 and Q-44, with
   m is 0, 1 or 2, limited by the number of available positions in Q to which a substituent Y can be connected, and
   each Y is independently selected from the group consisting of hydrogen, —CF₃, —CH₂CF₃, methyl, ethyl, fluorine, chlorine,
R¹ is hydrogen, and
A represents a phenyl group of formula (A1)

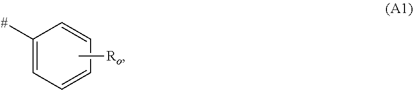

in which
# depicts the bond which connects A to the rest of the molecule,
o is 1 or 2, and
each R is independently selected from the group consisting of halogen, nitro, —OH, cyano, methyl and —CF₃, or
A represents a heterocycle of the formula (Het-1)

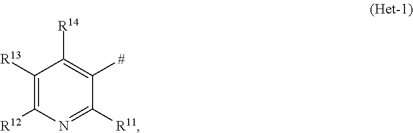

in which
# depicts the bond which connects A to the rest of the molecule,
R¹¹ is selected from the group consisting of hydrogen, halogen, methyl and —CF₃, and
R¹², R¹³ and R¹⁴, which may the same or different, are selected from the group consisting of hydrogen, halogen and —CF₃, or
A represents a heterocycle of the formula (Het-2)

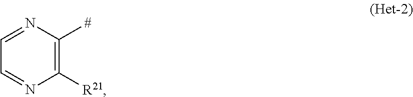

in which
# depicts the bond which connects A to the rest of the molecule, and
R²¹ is selected from the group consisting of hydrogen, halogen, methyl and —CF₃.

In another embodiment, the present invention provides compounds according to formula (I), wherein
n is 1,
X is selected from the group consisting of hydrogen, halogen, nitro, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
Q is selected from the group consisting of

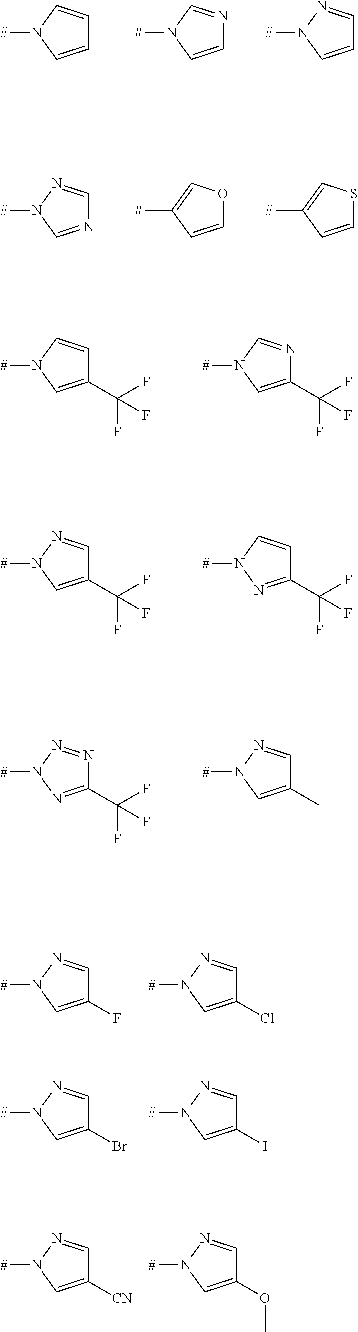

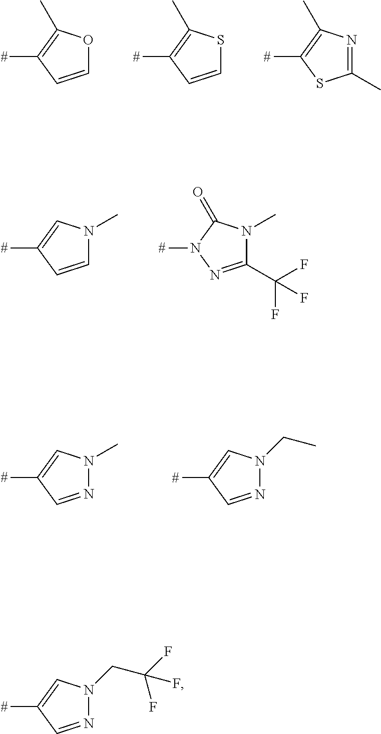

in which
# depicts the bond which connects Q to the rest of the molecule,
R¹ is hydrogen, and
A is selected from the group consisting of

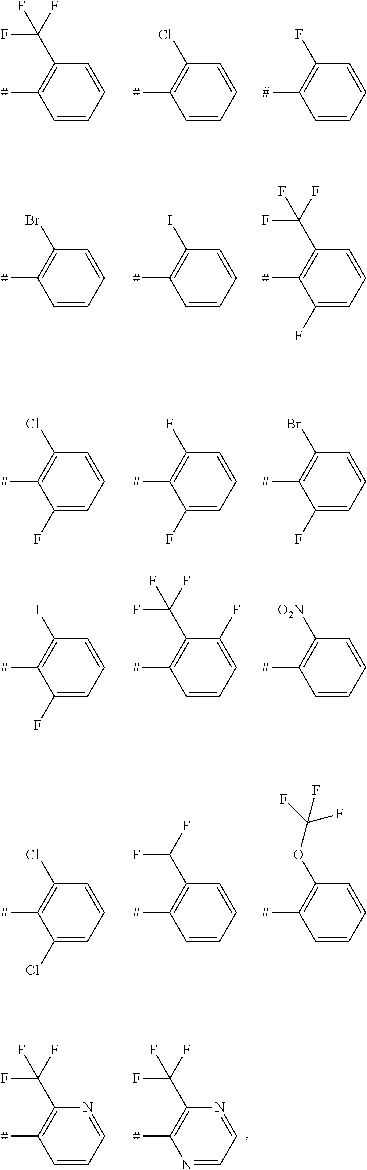

in which
# depicts the bond which connects A to the rest of the molecule.

In still another embodiment, the present invention provides compounds according to formula (I), wherein
n is 1,
X is selected from the group consisting of hydrogen or chlorine,
Q is selected from the group consisting of

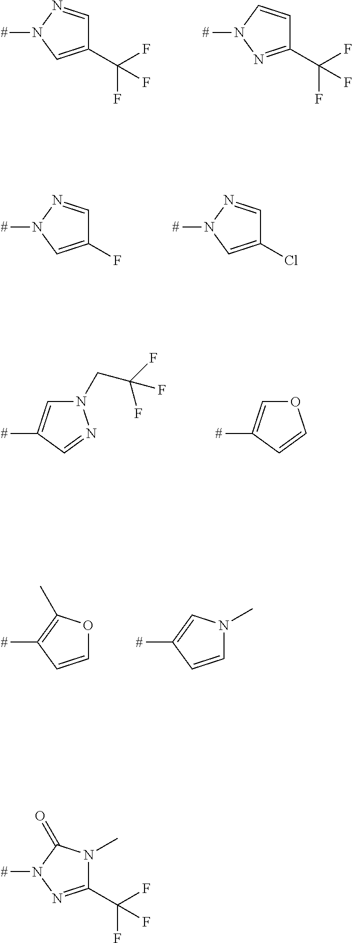

in which
# depicts the bond which connects Q to the rest of the molecule,
R¹ is hydrogen, and
A is selected from the group consisting of

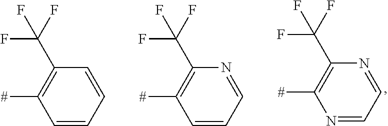

in which
# depicts the bond which connects A to the rest of the molecule.

In still another embodiment, the present invention provides compounds according to formula (I), wherein
n is 1,
X is selected from the group consisting of hydrogen or chlorine,
Q is selected from the group consisting of

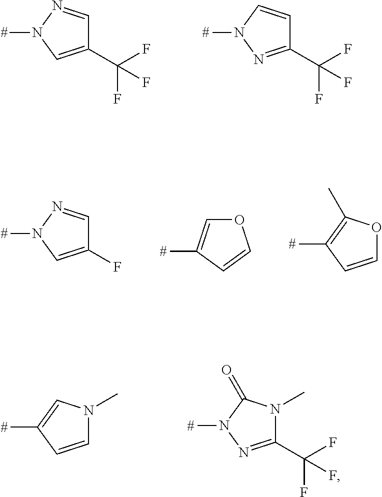

in which
# depicts the bond which connects Q to the rest of the molecule,
R¹ is hydrogen, and
A is selected from the group consisting of

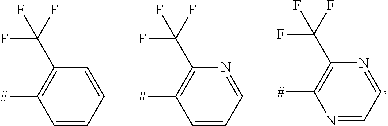

in which
# depicts the bond which connects A to the rest of the molecule.

In still another embodiment, the present invention provides compounds according to formula (I), wherein
n is 1,
X is chlorine,
Q is selected from the group consisting of

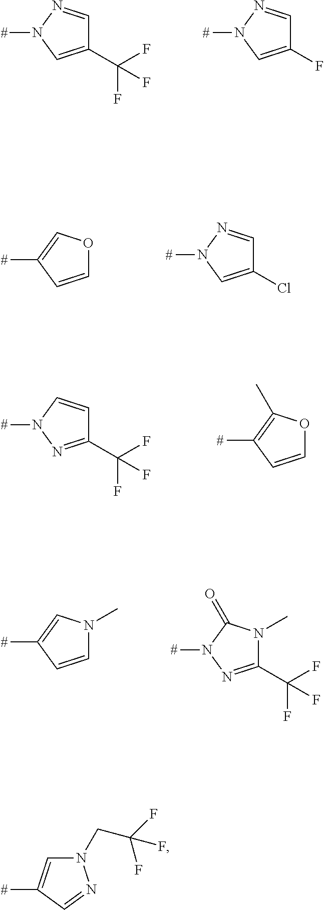

in which
# depicts the bond which connects Q to the rest of the molecule,
R¹ is hydrogen, and
A is selected from the group consisting of

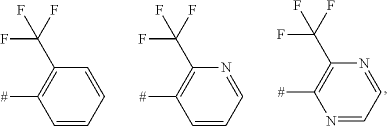

in which
# depicts the bond which connects A to the rest of the molecule.

In still another embodiment, the present invention provides compounds according to formula (I), wherein
n is 1,
X is chlorine,
Q is selected from the group consisting of

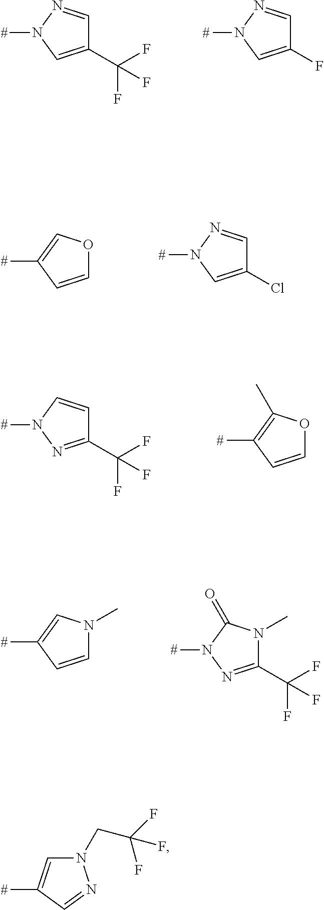

in which
# depicts the bond which connects Q to the rest of the molecule,
R¹ is hydrogen, and
A is

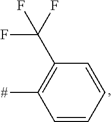

in which
# depicts the bond which connects A to the rest of the molecule.

In still another embodiment, the present invention provides compounds according to formula (I), wherein
n is 1,
X is chlorine,
Q is selected from the group consisting of

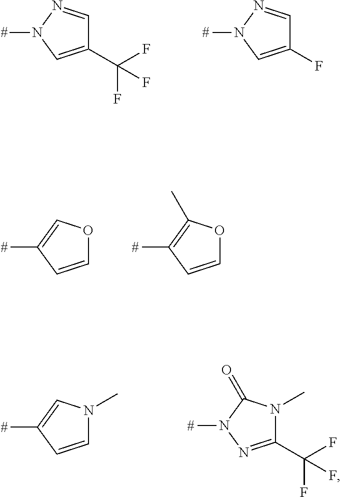

in which
# depicts the bond which connects Q to the rest of the molecule,
R¹ is hydrogen, and
A is

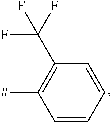

in which
# depicts the bond which connects A to the rest of the molecule.

The definitions of radicals indicated specifically in the respective combinations of radicals are replaced as desired irrespective of the particular combinations indicated for the radicals also by definitions of radicals of other combinations.

In a further embodiment, the present invention provides compounds of the following formula (I-1)

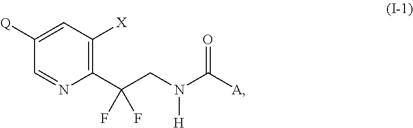

wherein
Q is a 5-membered ring selected from the group consisting of Q-1, Q-4, Q-6, Q-10, Q-21, Q-23, Q-24, Q-25, Q-27, Q-37, Q-41 and Q-44, with
   m is 0, 1 or 2, limited by the number of available positions in Q to which a substituent Y can be connected, and
   each Y is independently selected from the group consisting of hydrogen, halogen, C₁-C₄-alkyl and C₁-C₄-halogenoalkyl having 1 to 5 halogen atoms,
X is selected from the group consisting of fluorine, chlorine and trifluoro methyl, and
A represents a phenyl group of formula (A1)

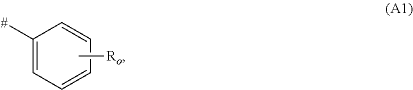

in which
# depicts the bond which connects A to the rest of the molecule,
o is 0, 1 or 2, and
each R is independently selected from the group consisting of halogen, nitro, —OH, cyano, C₁-C₄-alkyl, C₁-C₄-halogenoalkyl having 1 to 5 halogen atoms, C₃-C₆-cycloalkyl, C₁-C₄-alkoxy, C₁C₁-C₄-alkoxycarbonyl, —NH(C₁-C₄-alkyl), phenyl (optionally substituted by C₁-C₄-alkoxy) and phenoxy, or
A represents a heterocycle of the formula (Het-1)

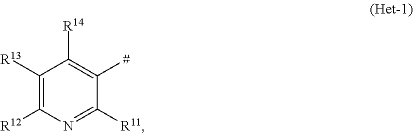

in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₈-alkyl, —S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and —S-phenyl (optionally substituted by halogen or C₁-C₄-alkyl), and
R¹², R¹³ and R¹⁴, which may the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, or
A represents a heterocycle of the formula (Het-2)

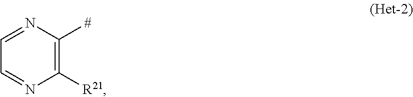

in which_{—}
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms,
with the proviso that if
   A is

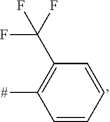

in which
# depicts the bond which connects A to the rest of the molecule, and
X is chlorine, then
Q is not one of the following

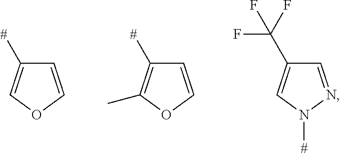

in which
# depicts the bond which connects Q to the rest of the molecule.

In another further embodiment, the present invention provides compounds of the following formula (I-1)

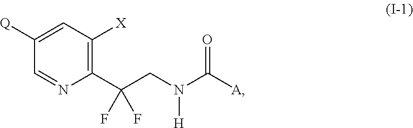

wherein
Q is a 5-membered ring selected from the group consisting of Q-1, Q-4, Q-6, Q-10, Q-21, Q-23, Q-24, Q-25, Q-27, Q-37, Q-41 and Q-44, with
   m is 0, 1 or 2, limited by the number of available positions in Q to which a substituent Y can be connected, and
   each Y is independently selected from the group consisting of hydrogen, halogen, C₁-C₄-alkyl and C₁-C₄-halogenoalkyl having 1 to 5 halogen atoms,
X is selected from the group consisting of fluorine, chlorine and trifluoro methyl, and
A represents a phenyl group of formula (A1)

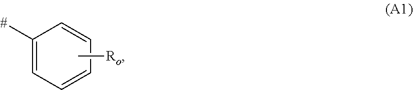

in which
# depicts the bond which connects A to the rest of the molecule,
o is 0, 1 or 2, and
each R is independently selected from the group consisting of halogen, nitro, —OH, cyano, C₁-C₄-alkyl, C₁-C₄-halogenoalkyl having 1 to 5 halogen atoms, C₃-C₆-cycloalkyl, C₁-C₄-alkoxy, C₁C₁-C₄-alkoxycarbonyl, —NH(C₁-C₄-alkyl), phenyl (optionally substituted by C₁-C₄-alkoxy) and phenoxy, or
A represents a heterocycle of the formula (Het-1)

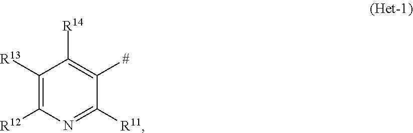

in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₈-alkyl, —S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and —S-phenyl (optionally substituted by halogen or C₁-C₄-alkyl), and
R¹², R¹³ and R¹⁴, which may the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, or
A represents a heterocycle of the formula (Het-2)

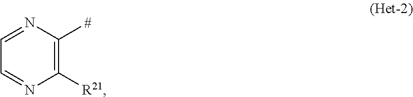

in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms,
for use in the control, treatment and/or prevention of infections with helminths in animals and humans.

The following embodiments that relate to formula (I-1) shall be understood as referring to such compounds according the present invention per se or to such compounds for use in the control, treatment and/or prevention of infections with helminths in animals and humans according to the present invention, or to both.

In still a further embodiment, the present invention provides compounds according to formula (I-1), wherein
A is selected from the group consisting of

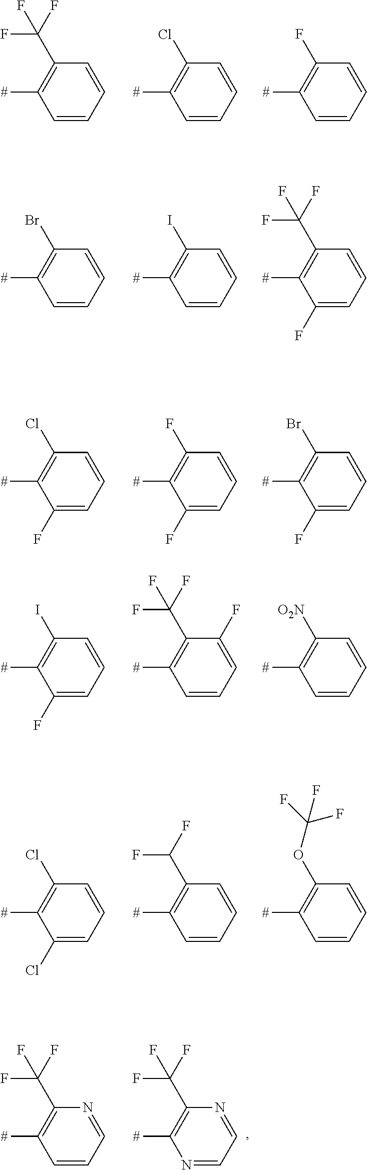

in which
# depicts the bond which connects A to the rest of the molecule, and
Q and X have the meaning as described before for formula (I-1).

In still a further embodiment, the present invention provides compounds according to formula (I-1), wherein
A is selected from the group consisting of

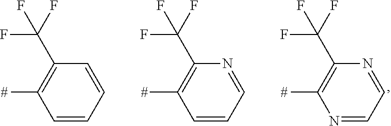

in which
# depicts the bond which connects A to the rest of the molecule, and
Q and X have the meaning as described before for formula (I-1).

In still a further embodiment, the present invention provides compounds according to formula (I-1), wherein
Q represents a 5-membered ring selected from the group consisting of Q-21, Q-23, Q-37 and Q-44, with
   m and Y having the meaning as described before for formula (I-1), and
X and A have the meaning as described before for formula (I-1).

In still a further embodiment, the present invention provides compounds according to formula (I-1), wherein
X is chlorine, and
Q and A have the meaning as described before for formula (I-1).

In still a further embodiment, the present invention provides compounds according to formula (I-1), wherein
A is selected from the group consisting of

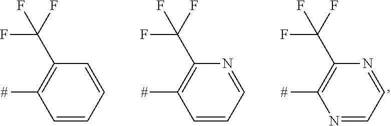

in which
# depicts the bond which connects A to the rest of the molecule,
X is chlorine, and
Q is selected from the group consisting of

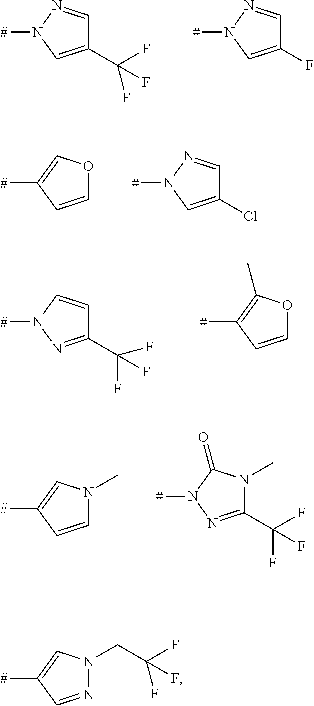

in which
# depicts the bond which connects Q to the rest of the molecule.

In still a further embodiment, the present invention provides compounds according to formula (I-1), wherein
A is

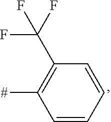

in which
# depicts the bond which connects A to the rest of the molecule,
X is chlorine, and
Q is selected from the group consisting of

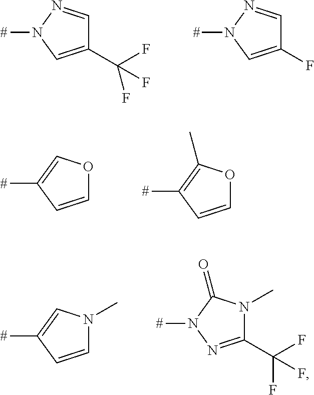

in which
# depicts the bond which connects Q to the rest of the molecule.

The definitions of radicals, and explanations, that are given above in general or in ranges of preference or further embodiments may be combined arbitrarily with one another, thus including combinations between the respective ranges and ranges of preference/embodiments. The definitions and explanations apply to the end products and also to the precursors and intermediates accordingly.

The invention further relates to a pharmaceutical composition comprising at least one compound of formula (I) according to anyone of the embodiments mentioned before.

The invention further relates to a pharmaceutical composition comprising at least one compound of formula (I) according to anyone of the embodiments mentioned before for the control, treatment and/or prevention of infections with helminths in animals and humans.

The invention further relates to the use of a compound of formula (I) of anyone of the embodiments mentioned before for the control, treatment and/or prevention of infections with helminths in animals and humans.

The invention further relates to the use of a pharmaceutical composition as mentioned before for the control, treatment and/or prevention of infections with helminths in animals and humans.

The invention further relates to the use of a compound of formula (I) of anyone of the embodiments mentioned before for the manufacturing of a medicament for the control, treatment and/or prevention of infections with helminths in animals and humans.

The invention further relates to a method for the control, treatment and/or prevention of infections with helminths in animals and humans, comprising the step of administering an effective amount of a compound of formula (I) of the embodiments mentioned before, or a pharmaceutical composition as mentioned before, to an animal or human in need thereof.

Saturated or unsaturated hydrocarbon radicals such as alkyl, alkanediyl or alkenyl may in each case, both alone and in conjunction with heteroatoms, as in alkoxy, for example, be—where possible—either straight-chain or branched.

Any substituted radicals may, unless indicated otherwise, be substituted one or more times, and the substituents in the case of multiple substitutions may be alike or different.

In the definitions of radicals that are stated as being preferred, halogen (halo) is fluoro, chloro, bromo and iodo, very preferably fluoro, chloro and bromo, and especially preferably fluoro and chloro.

Procedures and Methods

The synthesis of the compounds of the formula (I) can be performed according to or in analogy to scheme 1 (synthesis of compounds of formula (I-a)). The required starting materials are known or accessible via generally known procedures which are described in more detail in WO 2007/141009, WO 2013/064460 or WO 2014/004064.

The compounds of the formula (I-a) are synthesized by a coupling reaction. In the case of Q=N-bonded azoles, a copper-mediated process with an azole Q-H, copper(I)-oxide, potassium iodide, salicylaldoxime or trans-N,N-dimethylcyclohexanediamine as ligand in a solvent as acetonitrile or dioxane in the presence of a base as cesium carbonate or potassium carbonate may be used. In the case of Q=carbon-bonded heterocycles, a Suzuki-type coupling with the appropriate boronic acid (Q-B(OH)₂) or ester (Q-B(OR)₂) in the presence of a palladium catalyst and a base may be used.

Further compounds with Q=triazole of the formula (I-a) are accessible via conversion of the amine to the corresponding hydrazine via diazotation—reduction and subsequent triazole formation as described in US2011/0077410 A1. Compounds with Q=tetrazole are accessible via conversion of the amine into the corresponding nitrile via diazotation—cyanation and subsequent cycloaddition with azide.

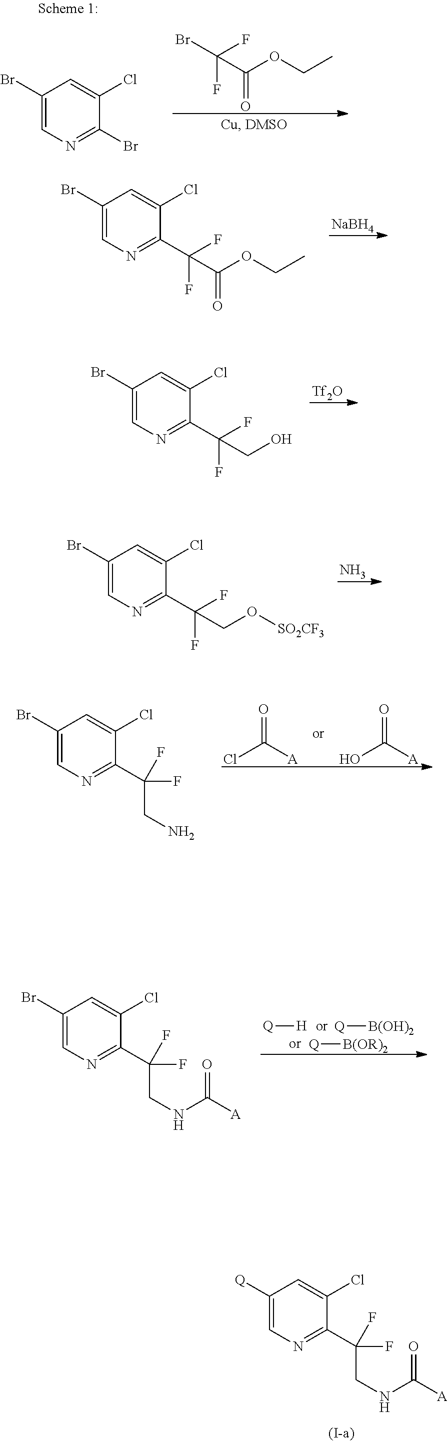

The compound according to the present invention can be prepared according to the processes described above. It will nevertheless be understood that, on the basis of his general knowledge and of available publications, the skilled worker will be able to adapt this method according to the specifics of each of the compounds, which it is desired to synthesize.

The compounds of the invention can be used as endoparasiticides. At least within the context of the present invention, the use as endoparasiticide shall comprise the use for the control, treatment and/or prevention of infections with helminths in animals and humans, preferably in non-human animals.

The compounds of the present invention act as anthelmintic agents against endoparasites in animals and humans.

In the veterinary field and in animal keeping, the administration of the active compounds according to the invention is carried out in the known manner directly or enterally, parenterally, dermally or nasally in the form of suitable preparations. Administration can be carried out prophylactically or therapeutically.

In the animal health field, i.e. in the field of veterinary medicine, the compounds according to the present invention are active against animal parasites, in particular endoparasites. The term endoparasite includes in particular helminths and protozoae, such as coccidia. The compounds of formula (I) are preferably active against helminths.

In the field of veterinary medicine the compounds according to the invention are suitable, with favourable warm blood toxicity, for controlling parasites, preferably helminths, which occur in animal breeding and animal husbandry in livestock, breeding, zoo, laboratory, experimental and domestic animals. They are active against all or specific stages of development of the parasites.

Agricultural livestock include, for example mammals, such as, sheep, goats, horses, donkeys, camels, buffaloes, rabbits, reindeers, fallow deers, and in particular cattle and pigs; or poultry such as turkeys, ducks, geese, and in particular chickens; or fish or crustaceans e.g. in aquaculture; or as the case may be insects such as bees.

Domestic animals include, for example mammals, such as hamsters, guinea pigs, rats, mice, chinchillas, ferrets or in particular dogs, cats; cage birds; reptiles; amphibians or aquarium fish.

According to a preferred embodiment, the compounds according to the invention are administered to mammals.

According to another preferred embodiment, the compounds according to the invention are administered to birds, namely cage birds or in particular poultry.

By using the active compounds according to the invention to control animal parasites, preferably helminths, it is intended to reduce or prevent illness, cases of deaths and performance reductions (in the case of meat, milk, wool, hides, eggs, honey and the like), so that more economical and simpler animal keeping is made possible and better animal well-being is achievable.

The term “control” or “controlling” as used herein with regard to the animal health field means that the active compounds are effective in reducing the numbers of the respective parasites in an animal infected with such parasites to innocuous levels. More specifically, “controlling”, as used herein, means that the active compound is effective in killing the respective parasites, inhibiting their growth, and/or inhibiting their proliferation.

Exemplary pathogenic endoparasites of humans and animals, which are helminths, include platyhelmintha (e.g. monogenea, cestodes and trematodes), nematodes, acanthocephala, and pentastoma. Additional exemplary helminths include, without any limitation:

### Monogenea: e.g.: Gyrodactylus spp., Dactylogyrus spp., Polystoma spp.

Cestodes: from the order of the Pseudophyllidea for example: Diphyllobothrium spp., Spirometra spp., Schistocephalus spp., Ligula spp., Bothridium spp., Diplogonoporus spp.
from the order of the Cyclophyllida for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.
Trematodes: from the class of the Digenea for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp. Metorchis spp., Heterophyes spp., Metagonimus spp.
Nematodes: from the order of the Trichinellida, for example: Trichuris spp., Capillaria spp., Paracapillaria spp., Trichomosoides spp., Trichinella spp., Eucoleus spp.
from the order of the Tylenchida for example: Micronema spp., Strongyloides spp.
from the order of the Rhabditina for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Necator spp., Ancylostoma spp., Uncinaria spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp. Parelaphostrongylus spp., Crenosoma spp., Paracrenosoma spp., Angiostrongylus spp., Aelurostrongylus spp., Filaroides spp., Parafilaroides spp., Oslerus spp., Trichostrongylus spp., Haemonchus spp., Ostertagia spp., Teladorsagia spp., Marshallagia spp., Cooperia spp., Nematodirus spp., Hyostrongylus spp., Obeliscoides spp., Amidostomum spp., Ollulanus spp.; Heligmosomoides spp., Nippostrongylus spp.
from the order of the Spirurida for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp., Spirocerca spp.
Acantocephala: from the order of the Oligacanthorhynchida, for example: Macracanthorhynchus spp., Prosthenorchis spp.; from the order of the Polymorphida, for example: Filicollis spp.; from the order of the Moniliformida, for example: Moniliformis spp.
from the order of the Echinorhynchida, for example: Acanthocephalus spp., Echinorhynchus spp., Leptorhynchoides spp.
Pentastoma: from the order of the Porocephalida for example Linguatula spp.

Thus, one embodiment of the present invention refers to compounds according to the invention for use as a medicament.

Another aspect refers to compounds according to the invention for use as an antiendoparasitical agent, in particular an anthelminthic agent. For example, compounds according to the invention can be used as an antiendoparasitical agent, in particular an anthelminthic agent, e.g., in animal husbandry, in animal breeding, in animal housing, in the hygiene sector.

In the present context of the animal health or veterinary field, the term “treatment” includes prophylactic, metaphylactic or therapeutic treatment. In a particular embodiment, for the animal health field, mixtures with other anthelmintics are also provided.

Exemplary mixing partners include, without any limitation:

Anthelmintic actives, including nematicidal, trematicidal and cestocidal actives:
From the class of macrocyclic lactones, for example:
abamectin, doramectin, emamectin, eprinomectin, ivermectin, milbemycin, moxidectin, nemadectin, selamectin;
from the class of benzimidazoles and probenzimidazoles, for example:
albendazole, albendazole sulfoxide, cambendazole, cyclobendazole, febantel, fenbendazole, flubendazole, mebendazole, netobimin, oxfendazole, oxibendazole, parbendazole, thiabendazole, thiophanate, triclabendazole;
from the class of cyclooctadepsipeptides, for example:
emodepside, PF1022;
from the class of aminoacetonitrile derivatives, for example:
monepantel;
from the class of tetrahydropyrimidines, for example:
morantel, pyrantel, oxantel;
from the class of imidazothiazoles, for example:
butamisole, levamisole, tetramisole;
from the class of salicylanilides, for example:
bromoxanide, brotianide, clioxanide, closantel, niclosamide, oxyclozanide, rafoxanide, tribromsalan;
from the class of paraherquamides, for example:
derquantel, paraherquamide;
from the class of aminophenylamidines, for example:
amidantel, deacylated amidantel (dAMD), tribendimidine;
from the class of organophosphates, for example:
coumaphos, crufomate, dichlorvos, haloxon, naphthalofos, trichlorfon;
from the class of substituted phenols, for example:
bithionole, disophenol, hexachlorophen, niclofolan, meniclopholan, nitroxynil;
from the class of piperazinones, for example:
praziquantel, epsiprantel;
from the class of carbanilides, for example:
imidocarb;
from the class of quinazolinone alkaloid, for example:
halofuginon;
from the class of sulfonamides, for example:
sulfaclozin;
from the class of triazines, for example:
diclazuril, toltrazuril;
from diverse other classes, for example:
amoscanate, bephenium, bunamidine, clonazepam, clorsulon, diamfenetide, dichlorophen, diethylcarbamazine, emetine, hetolin, hycanthone, lucanthone, miracil, mirasan, niclosamide, niridazole, nitroxynile, nitroscanate, oltipraz, omphalotin, oxamniquine, paromomycin, piperazine, resorantel.

All named mixing partners can, if their functional groups enable this, optionally form salts with suitable bases or acids.

In another particular embodiment, for the animal health field, mixtures with ectoparasiticides are also provided.

Exemplary mixing partners include, without any limitation:

from the class of amidine derivatives, for example:
amitraz, chlormebuform, cymiazole, demiditraz;
from the class of arylisoxazolines, not excluding related classes with pyrroline or pyrrolidine moiety replacing the isoxazoline ring, for example:
afoxolaner, fluralaner;
from the class of bacillus thuringiensis strains, for example:
bacillus thuringiensis strains;
from the class of benzoylureas, for example:
bistrifluron, chlofluazuron, chlorfluazuron, diflubenzuron, fluazuron, flucycloxuron, flufenoxuron, hexaflumuron, lufenuron, novaluron, noviflumuron, penfluron, teflubenzuron, triflumuron;
from the class of beta-ketonitrile derivatives, for example:
cyenopyrafen, cyflumetofen;
from the class of carbamates, for example:
alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, formparanate, furathiocarb, isoprocarb, metam-sodium, methiocarb, methomyl, metolcarb, oxamyl, pirimicarb, promecarb, propoxur, thiodicarb, thiofanox, triazamate, trimethacarb, xmc, xylylcarb;
from the class of chloronicotinyls, for example:
acetamiprid, clothianidin, dinotefuran, flupyradifurone, imidacloprid, nicotine, nitenpyram, nithiazine, thiacloprid, thiamethoxam;
from the class of diacylhydrazines, for example:
chromafenozide, halofenozide, methoxyfenozide, tebufenozide;
from the class of diamides, for example:
chlorantraniliprole, cyantraniliprole;
from the class of dicarboxamides, for example:
flubendiamide;
from the class of dinitrophenols, for example:
binapacyrl, dinobuton, dinocap, dnoc;
from the class of feeding inhibitors, for example:
cryolite, flonicamid, pymetrozine;
from the class of fumigants, for example:
aluminium phosphide, methyl bromide, sulphuryl fluoride;
from the class of halogenated carbonhydrogen compounds (hch), for example:
ddt, methoxychlor;
from the class of macrocyclic lactones, for example:
moxidectin, emamectin benzoate, latidectin, lepimectin;
from the class of microorganisms, for example:
bacillus spec., beauveria spec., metarrhizium spec., paecilomyces spec., verticillium spec.;
from the class of mite growth inhibitors, for example:
amidoflumet, benclothiaz, benzoximate, bifenazate, bromopropylate, chlordimeform, chlorobenzilate, chloropicrin, clofentezine, clothiazoben, cycloprene, dicyclanil, etoxazole, fenoxacrim, fentrifanil, flubenzimine, flufenerim, flutenzin, gossyplure, hexythiazox, hydramethylnone, japonilure, metoxadiazone, petroleum, potassium oleate, pyridalyl, quinomethionate, tetrasul, triarathene;
from the class of natural products, for example:
codlemone, essential oils, thuringiensin;
from the class of neem components, for example:
azadirachtin a;
from the class of nereistoxin analogues, for example:
bensultap, cartap, sulfoxaflor, thiocyclam, thiocyclam hydrogen oxalate, thiosultap sodium, thiosultap-sodium;
from the class of organic acids, for example:
formic acid, oxalic acid;
from the class of organochlorines, for example:
camphechlor, chlordane, endosulfan, gamma-hch, hch, heptachlor, lindane;
from the class of organophosphates, for example:
acephate, aromfenvinfos (-methyl), aromophos-ethyl, autathiofos, azamethiphos, azinphos (-methyl, -ethyl), cadusafos, carbophenothion, chlorethoxyfos, chlorfenvinphos, chlormephos, chlorpyrifos (-methyl/-ethyl), cyanofenphos, cyanophos, demeton-s-methyl, demeton-s-methylsulphone, dialifos, diazinon, dichlofenthion, dichlorvos/ddvp, dicrotophos, dimethoate, dimethylvinphos, dioxabenzofos, disulfoton, epn, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl o-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidone, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion;
from the class of organotin compounds, for example:
azocyclotin, cyhexatin, fenbutatin-oxide;
from the class of other decouplers, for example:
sulfluramid;
from the class of other inhibitors of cuticle development, for example:
buprofezin, cyromazine;
from the class of other inhibitors of cuticle development, for example:
buprofezin, cyromazine;
from the class of others, for example:
chinomethionat, pyrifluquinazon;
from the class of oxadiazines, for example:
indoxacarb;
from the class of phenylpyrazoles, for example:
acetoprole, ethiprole, fipronil, pyrafluprole, pyriprole, vaniliprole;
from the class of pyrethroids, for example:
acrinathrin, allethrin (d-cis-trans, d-trans-), beta-cyfluthrin, bifenthrin, bioallethrin, bioallethrin-s-cyclopentyl-isomer, bioethanomethrin, biopermethrin, bioresmethrin, chlovaporthrin, cis-cypermethrin, cis-permethrin, cis-resmethrin, clocythrin, cycloprothrin, cyfluthrin, cyhalothrin (lambda-), cypermethrin (alpha-, beta-, theta-, zeta-), cyphenothrin, deltamethrin, empenthrin (1r-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1r-trans isomer), prallethrin, profluthrin, protrifenbute, pyresmethrin, pyrethrins (pyrethrum), resmethrin, ru 15525, silafluofen, tau-fluvalinate, tefluthrin, terallethrin, tetramethrin (-1r-isomer), tralomethrin, transfluthrin, zxi 8901;
from the class of pyrroles, for example:
chlorfenapyr;
from the class of quinones, for example:
acequinocyl;
from the class of rotenone, for example:
rotenone;
from the class of semicarbazones, for example:
metaflumizone;
from the class of spinosynes, for example:
spinetoram, spinosad;
from the class of tetronic and tetramic acids, for example:
spirodiclofen, spiromesifen, spirotetramat;
from the class of nereistoxin analogues, for example:
bensultap, cartap, sulfoxaflor, thiocyclam, thiocyclam hydrogen oxalate, thiosultap sodium, thiosultap-sodium;
from diverse other classes, for example:
amoscanate, bephenium, bunamidine, clonazepam, clorsulon, diamfenetide, dichlorophen, diethylcarbamazine, emetine, hetolin, hycanthone, lucanthone, miracil, mirasan, niclosamide, niridazole, nitroxynile, nitroscanate, oltipraz, omphalotin, oxamniquine, paromomycin, piperazine, resorantel.

Salts like hydrochlorides, tartrates, citrates, embonates/pamoates or benzoates are included.

### PREPARATION EXAMPLES

¹H-NMR-data were determined with a Bruker Avance 400 equipped with a flow cell (60 μl volume) or with a Bruker AVIII 400 equipped with 1.7 mm cryo-CPTCI probe head or with a Bruker AVII 600 (600.13 MHz) equipped with a cyroTCI probe head or with a Bruker AVIII 600 (601.6 MHz) equipped with a cryo CPMNP probe head with tetramethylsilane as reference (0.0) and the solvents CD₃CN, CDCl₃, D₆-DMSO.

NMR-data of selected examples are listed in classic format (chemical shift δ, multiplicity, number of hydrogen atoms) or as NMR-peak-lists.

The NMR spectra of the steps of preparation example 1 and the NMR spectra of examples 1-6 have been measured on a Varian 400 MHz Mercury Plus.

### Preparation Example 1

### Step 1

### Synthesis of 2-(5-bromo-3-chloropyridin-2-yl)-2,2-difluoroethanamine was performed in analogy to WO 2013/064460 A1

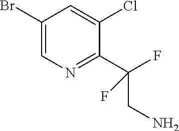 [SMILES: NCC(F)(F)c1ncc(Br)cc1Cl]

¹H-NMR (400 MHz, d6-DMSO); δ 8.78 (d, J=1.6 Hz, 1H), 8.52 (d, J=2.0 Hz, 1H), 3.37 (t, J=14.8 Hz, 2H), 1.72 (s, 2H).

### Step 2

### Synthesis of N-[2-(5-bromo-3-chloropyridin-2-yl)-2,2-difluoroethyl]-2-(trifluoromethyl)benzamide

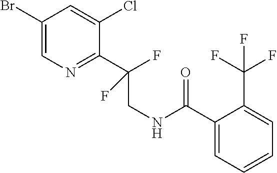 [SMILES: O=C(NCC(F)(F)c1ncc(Br)cc1Cl)c1ccccc1C(F)(F)F]

To a solution of 2-(5-bromo-3-chloropyridin-2-yl)-2,2-difluoroethanamine (2.56 g, 1.03 eq.) in dichloromethane (50 ml) at room temperature, triethylamine (3.38 ml, 3.0 eq.) and 2-trifluoromethyl benzoic acid chloride (1.68 g, 1.0 eq.) were added and stirred over night. After completion of reaction, the reaction mixture was diluted with water and extracted with dichloromethane. The solvent of the combined organic layers was evaporated under reduced pressure. The residue was purified by silica gel chromatography (cyclohexane/ethyl acetate) to yield 2.86 g (68.5%) as off-white solid.

¹H-NMR (400 MHz, d6-DMSO); δ 8.97 (t, 1H, NH), 8.80 (d, 1H), 8.56 (d, 1H), 7.77-7.63 (m, 3H), 7.45 (d, 1H), 4.28-4.19 (m, 2H).

### Step 3

### Synthesis of N-[2-[3-chloro-5-[4-(trifluoromethyl)pyrazol-1-yl]-2-pyridyl]-2,2-difluoro-ethyl]-2-(trifluoromethyl)benzamide (example 3)

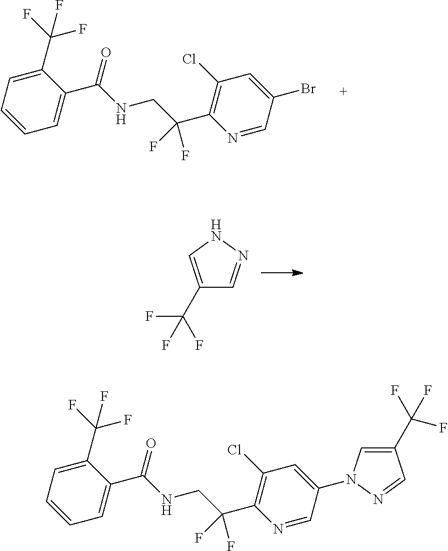 [SMILES: O=C(NCC(F)(F)c1ncc(Br)cc1Cl)c1ccccc1C(F)(F)F ; FC(F)(F)c1cn[nH]c1 ; O=C(NCC(F)(F)c1ncc(-n2cc(C(F)(F)F)cn2)cc1Cl)c1ccccc1C(F)(F)F]

170 mg (0.38 mmol) N-[2-(5-bromo-3-chloropyridin-2-yl)-2,2-difluoroethyl]-2-(trifluoromethyl) benzamide (from step 2) and 62.6 mg (0.46 mmol) 4-(trifluoromethyl)-1H-pyrazole were dissolved in 5 mL acetonitrile. Thereafter, 5.48 mg copper(II) oxide (0.03 mmol), 187.3 mg (0.57 mmol) cesium carbonate and 10.5 mg (0.07 mmol) salicyl aldoxime were added and heated in a sealed vial at 100° C. for 24 hours. The reaction mixture was filtered over a silica gel—sodium sulfate cartridge, the solvents were evaporated and the crude product was purified by preparative HPLC to afford 50 mg (24.4%) of the title compound as off-white solid.

¹H-NMR (400 MHz, d6-DMSO); 9.45 (s, 1H), 9.21 (s, 1H), 9.01 (t, 1H, NH), 8.69 (d, 1H), 8.34 (s, 1H), 7.78-7.63 (m, 3H), 7.47 (d, 1H), 4.34-4.25 (m, 2H).

According to the methods described above, the following compounds of general formula (I) have been prepared.

TABLE 1
Compounds of formula (I)

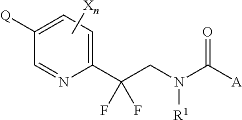

Q, X, n, A, R¹ as defined by each individual structure.

| Example | | logP ^{a)} | M + 1 | |
|---|---|---|---|---|
| No. | Formula | (HCOOH) | (LC/MS) | ¹H-NMR |
| 1 | 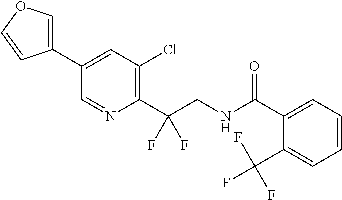 [SMILES: O=C(NCC(F)(F)c1ncc(-c2ccoc2)cc1Cl)c1ccccc1C(F)(F)F] | 3.21 | 430.1 | NMR peak list |
| 2 | 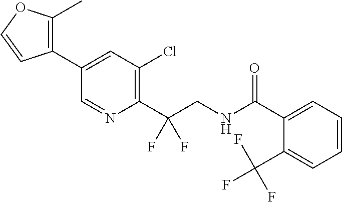 [SMILES: Cc1occc1-c1cnc(C(F)(F)CNC(=O)c2ccccc2C(F)(F)F)c(Cl)c1] | 3.53 | 444.1 | NMR peak list |
| 3 | 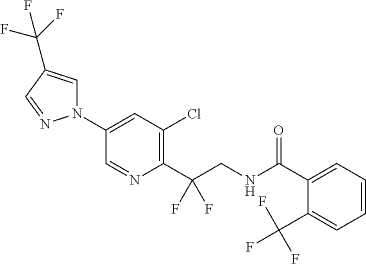 [SMILES: O=C(NCC(F)(F)c1ncc(-n2cc(C(F)(F)F)cn2)cc1Cl)c1ccccc1C(F)(F)F] | 3.62 | 499.0 | 400 MHz, D₆-DMSO: 9.45 (s, 1H), 9.21 (s, 1H), 9.01 (t, 1H, NH), 8.69 (d, 1H), 8.34 (s, 1H), 7.78- 7.63 (m, 3H), 7.47 (d, 1H), 4.34-4.25 (m, 2H). |
| 4 | 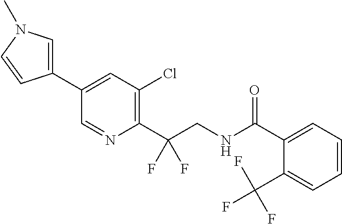 [SMILES: Cn1ccc(-c2cnc(C(F)(F)CNC(=O)c3ccccc3C(F)(F)F)c(Cl)c2)c1] | 3.22 | 444.0 | NMR peak list |
| 5 | 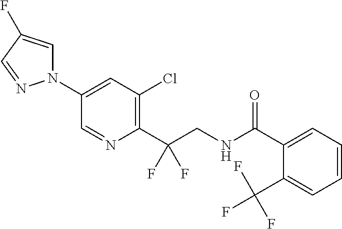 [SMILES: O=C(NCC(F)(F)c1ncc(-n2cc(F)cn2)cc1Cl)c1ccccc1C(F)(F)F] | 3.12 | 449.1 | NMR peak list |
| 6 | 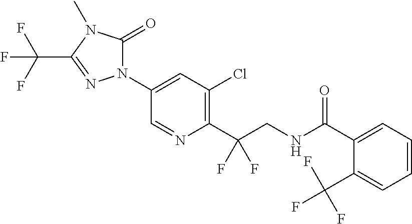 [SMILES: Cn1c(C(F)(F)F)nn(-c2cnc(C(F)(F)CNC(=O)c3ccccc3C(F)(F)F)c(Cl)c2)c1=O] | 3.32 | 530.0 | NMR peak list |
| 7 | 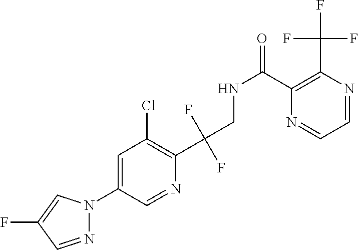 [SMILES: O=C(NCC(F)(F)c1ncc(-n2cc(F)cn2)cc1Cl)c1nccnc1C(F)(F)F] | 2.88 | 451.0 | NMR peak list |
| 8 | 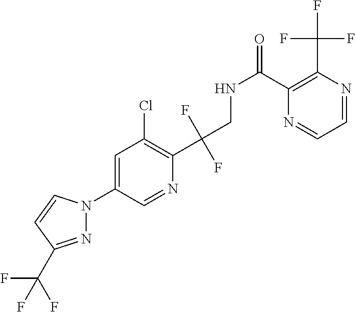 [SMILES: O=C(NCC(F)(F)c1ncc(-n2ccc(C(F)(F)F)n2)cc1Cl)c1nccnc1C(F)(F)F] | 3.49 | 501.0 | NMR peak list |
| 9 | 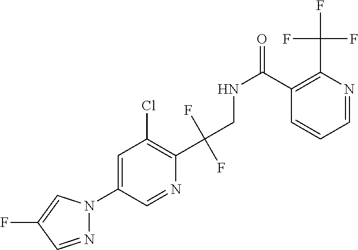 [SMILES: O=C(NCC(F)(F)c1ncc(-n2cc(F)cn2)cc1Cl)c1cccnc1C(F)(F)F] | 2.7 | 450.0 | NMR peak list |
| 10 | 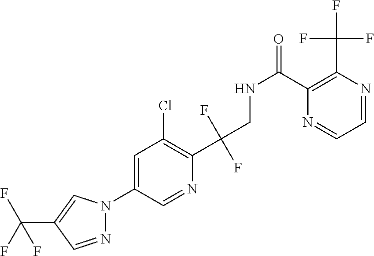 [SMILES: O=C(NCC(F)(F)c1ncc(-n2cc(C(F)(F)F)cn2)cc1Cl)c1nccnc1C(F)(F)F] | 3.43 | 501.0 | NMR peak list |
| 11 | 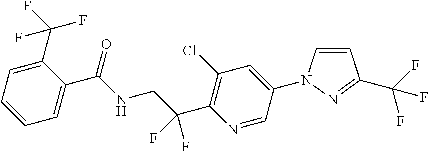 [SMILES: O=C(NCC(F)(F)c1ncc(-n2ccc(C(F)(F)F)n2)cc1Cl)c1ccccc1C(F)(F)F] | 3.63 | 499.0 | NMR peak list |
| 12 | 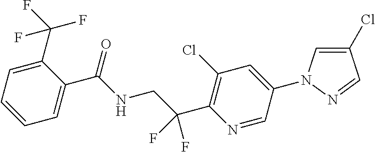 [SMILES: O=C(NCC(F)(F)c1ncc(-n2cc(Cl)cn2)cc1Cl)c1ccccc1C(F)(F)F] | 3.5 | 465.0 | NMR peak list |
| 13 | 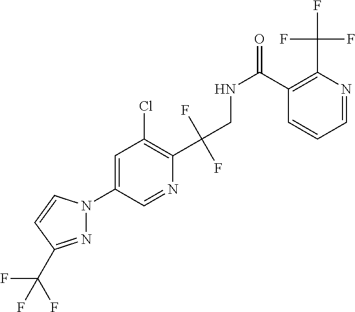 [SMILES: O=C(NCC(F)(F)c1ncc(-n2ccc(C(F)(F)F)n2)cc1Cl)c1cccnc1C(F)(F)F] | 3.27 | 500.0 | NMR peak list |
| 14 | 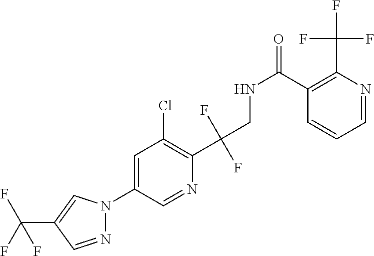 [SMILES: O=C(NCC(F)(F)c1ncc(-n2cc(C(F)(F)F)cn2)cc1Cl)c1cccnc1C(F)(F)F] | 3.24 | 500.1 | NMR peak list |
| 15 | 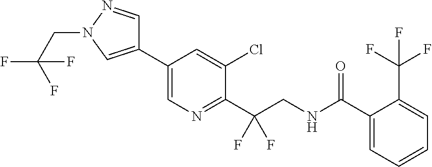 [SMILES: O=C(NCC(F)(F)c1ncc(-c2cnn(CC(F)(F)F)c2)cc1Cl)c1ccccc1C(F)(F)F] | 3.1 | 513.1 | NMR peak list |
| 16 | 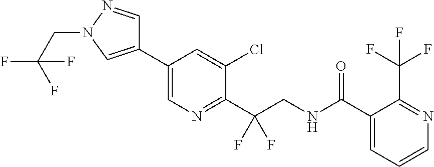 [SMILES: O=C(NCC(F)(F)c1ncc(-c2cnn(CC(F)(F)F)c2)cc1Cl)c1cccnc1C(F)(F)F] | 2.66 | 513.9 | NMR peak list |
| 17 | 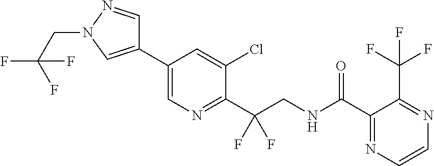 [SMILES: O=C(NCC(F)(F)c1ncc(-c2cnn(CC(F)(F)F)c2)cc1Cl)c1nccnc1C(F)(F)F] | 2.85 | 515.0 | NMR peak list |
| 18 | 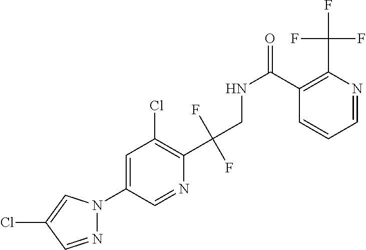 [SMILES: O=C(NCC(F)(F)c1ncc(-n2cc(Cl)cn2)cc1Cl)c1cccnc1C(F)(F)F] | 3.07 | 465.9 | NMR peak list |

LC-MS

Measurement of Log P values was performed according to EEC directive 79/831 Annex V.A8 by HPLC (High Performance Liquid Chromatography) on reversed phase columns with the following methods:
[^{a}] Log P value is determined by measurement of LC-UV, in an acidic range, with 0.1% formic acid in water and acetonitrile as eluent (linear gradient from 10% acetonitrile to 95% acetonitrile).
[^{b}] Log P value is determined by measurement of LC-UV, in a neutral range, with 0.001 molar ammonium acetate solution in water and acetonitrile as eluent (linear gradient from 10% acetonitrile to 95% acetonitrile).

Calibration was done with straight-chain alkan-2-ones (with 3 to 16 carbon atoms) with known Log P values (measurement of Log P values using retention times with linear interpolation between successive alkanones). Lambda-max-values were determined using UV-spectra from 200 nm to 400 nm and the peak values of the chromatographic signals.

In table 1, M+1 (or M+H) means the molecular ion peak, plus or minus 1 a.m.u. (atomic mass unit) respectively, as observed in mass spectroscopy by electrospray ionization (ESI + or −).

¹H-NMR Data

¹H-NMR-data were determined with a Bruker Avance 400 equipped with a flow cell (60 μl volume) or with a Bruker AVIII 400 equipped with 5 mm cryo-CPTCI probe head or with a Bruker AVII 600 (600.13 MHz) equipped with a cyroTCI probe head or with a Bruker AVIII 600 (5 601.6 MHz) equipped with a 5 mm cryo CPMNP probe head with tetramethylsilane as reference (0.0) and the solvents CD₃CN, CDCl₃, or D₆-DMSO.

NMR Peak Lists

¹H-NMR data of selected examples are written in form of ¹H-NMR-peak lists. The δ-value in ppm and the signal intensity are listed to each signal peak in round brackets. Between the δ-value—signal intensity pairs are semicolons as delimiters.

The peak list of an example has therefore the form:

δ₁ (intensityi); δ₂ (intensity₂); . . . ; δᵢ (intensityi); . . . ; δₙ (intensityₙ)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

Tetramethylsilane and/or the chemical shift of the used solvent, especially in the case of spectra measured in DMSO (dimethylsulfoxide), have been used for calibrating. Therefore, tetramethylsilane peak can occur but not necessarily in NMR peak lists.

The ¹H-NMR peak lists are similar to classical ¹H-NMR prints and contain therefore usually all peaks, which are listed at classical NMR-interpretation.

Additionally they can show like classical ¹H-NMR prints signals of solvents, stereoisomers of the target compounds, which are also object of the invention, and/or peaks of impurities.

The usual peaks of solvents, for example peaks of DMSO in D₆-DMSO and the peak of water, are given in the ¹H-NMR peak lists to show compound signals in the delta-range of solvents and/or water. They have usually on average a high intensity.

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore, their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical ¹H-NMR interpretation.

Further details of NMR-data description with peak lists can be found in the publication “Citation of NMR Peaklist Data within Patent Applications” (cf. Research Disclosure Database Number 564025, 2011, 16 Mar. 2011 or http://www.rdelectronic.co.uk/rd/free/rd564025.pdf).

Table with NMR Peaklists

Example 1: ¹H-NMR(400.0 MHz, d6-DMSO):
δ = 8.985(3.3); 8.971(6.8); 8.955(3.6); 8.942(14.3); 8.938(14.6); 8.523(15.6); 8.492(0.3); 8.412(14.2); 8.408(13.9);
8.317(0.8); 7.936(0.3); 7.857(9.5); 7.853(16.0); 7.849(9.9); 7.799(0.4); 7.781(7.3); 7.761(10.0); 7.747(3.3);
7.729(8.2); 7.710(5.7); 7.668(5.8); 7.649(7.3); 7.630(2.8); 7.472(8.6); 7.453(7.6); 7.215(13.2); 7.212(13.1);
4.326(3.3); 4.310(3.3); 4.289(7.5); 4.273(7.3); 4.251(3.9); 4.235(3.5); 3.345(159.1); 3.219(0.4); 3.205(0.3);
2.996(0.5); 2.711(0.5); 2.676(1.8); 2.672(2.3); 2.667(1.7); 2.542(130.3); 2.507(297.5); 2.503(386.0); 2.498(282.1);
2.434(0.7); 2.427(0.6); 2.406(0.3); 2.376(0.3); 2.368(0.7); 2.334(1.8); 2.329(2.3); 2.325(1.8); 2.292(0.5); 2.048(0.4);
1.259(0.5); 1.235(2.1); 0.146(0.5); 0.008(3.8); 0.000(101.9); −0.008(3.8); −0.021(0.4); −0.150(0.6)
Example 2: ¹H-NMR(400.0 MHz, d6-DMSO):
δ = 8.994(3.3); 8.979(7.2); 8.963(3.3); 8.757(14.2); 8.753(14.1); 8.439(0.4); 8.317(1.0); 8.177(14.4); 8.172(14.0);
7.872(0.4); 7.855(0.5); 7.782(7.1); 7.762(9.7); 7.750(3.4); 7.731(7.9); 7.713(5.8); 7.684(15.7); 7.680(16.0);
7.669(6.1); 7.650(7.2); 7.631(2.8); 7.569(0.3); 7.553(0.7); 7.533(0.5); 7.520(0.4); 7.504(0.5); 7.483(8.2); 7.464(7.2);
6.953(14.6); 6.948(14.3); 4.340(3.1); 4.325(3.4); 4.303(7.3); 4.288(7.0); 4.266(3.7); 4.251(3.4); 3.338(161.3);
3.229(0.5); 3.170(0.5); 2.712(0.4); 2.676(1.7); 2.671(2.5); 2.542(112.1); 2.507(320.9); 2.503(392.5); 2.498(281.0);
2.494(136.2); 2.447(0.8); 2.418(0.5); 2.395(0.4); 2.367(0.7); 2.346(0.6); 2.334(1.7); 2.329(2.3); 2.325(1.8);
2.291(0.5); 2.271(0.4); 2.186(0.4); 1.740(0.4); 1.685(0.3); 1.258(0.5); 1.235(1.9); 0.146(0.4); 0.008(4.2);
0.000(95.8); −0.008(3.4); −0.150(0.5)
Example 3: ¹H-NMR(400.0 MHz, d6-DMSO):
δ = 9.522(0.5); 9.471(0.6); 9.453(0.8); 9.439(11.7); 9.328(0.4); 9.214(11.7); 9.208(11.7); 9.156(0.5); 9.094(0.4);
9.028(2.6); 9.012(5.4); 8.997(2.6); 8.970(0.3); 8.953(0.4); 8.847(0.6); 8.785(0.3); 8.715(0.6); 8.710(0.7);
8.689(11.1); 8.684(10.9); 8.429(0.8); 8.399(16.0); 8.316(2.0); 8.173(0.3); 8.109(0.3); 7.992(0.7); 7.960(0.4);
7.938(0.5); 7.879(0.8); 7.856(0.4); 7.841(0.4); 7.817(0.5); 7.801(0.5); 7.780(5.4); 7.759(7.6); 7.732(6.2); 7.714(4.4);
7.670(4.4); 7.650(5.5); 7.631(2.1); 7.523(0.4); 7.517(0.4); 7.500(0.5); 7.473(6.4); 7.454(5.6); 5.757(1.3); 4.338(2.5);
4.322(2.7); 4.301(5.7); 4.286(5.4); 4.264(3.0); 4.249(2.7); 4.033(0.4); 4.009(0.3); 3.551(0.4); 3.483(0.5); 3.463(0.7);
3.447(0.6); 3.438(0.5); 3.424(1.0); 3.406(1.1); 3.390(2.1); 3.339(1821.8); 3.306(2.8); 3.300(2.3); 3.283(0.7);
3.275(0.8); 3.264(1.0); 3.250(1.0); 3.169(0.3); 2.891(0.9); 2.731(0.8); 2.677(2.5); 2.672(3.5); 2.668(2.5); 2.592(0.3);
2.525(9.1); 2.512(212.0); 2.507(427.4); 2.503(558.1); 2.498(396.7); 2.494(186.4); 2.469(1.1); 2.429(0.4);
2.334(2.6); 2.330(3.4); 2.325(2.5); 1.386(1.9); 1.235(0.6); 1.175(0.7); 1.166(1.2); 1.150(0.6); 0.146(2.0); 0.021(0.7);
0.018(1.0); 0.008(20.0); 0.000(511.9); −0.009(17.4); −0.025(0.5); −0.034(0.4); −0.040(0.4); −0.150(2.0)
Example 4: 1H-NMR (400.0 MHz, d6-DMSO):
δ = 8.956 (0.8); 8.940 (1.6); 8.925 (0.8); 8.786 (3.2); 8.781 (3.3); 8.160 (3.3); 8.156 (3.3); 7.781 (1.6); 7.762 (2.3);
7.746 (0.7); 7.728 (1.9); 7.709 (1.3); 7.666 (1.3); 7.647 (1.7); 7.628 (0.7); 7.538 (1.8); 7.533 (3.1); 7.529 (1.9); 7.479
(1.9); 7.460 (1.7); 6.845 (1.7); 6.839 (2.9); 6.833 (1.9); 6.650 (2.0); 6.646 (2.5); 6.644 (2.5); 6.639 (1.9); 4.312 (0.7);
4.296 (0.7); 4.275 (1.7); 4.259 (1.6); 4.237 (0.9); 4.222 (0.8); 3.934 (0.5); 3.665 (16.0); 3.331 (14.5); 2.525 (0.4);
2.507 (18.5); 2.502 (24.0); 2.498 (17.8); 1.989 (0.4); 1.071 (4.2); 0.008 (0.7); 0.000 (19.0); −0.008 (0.8)
Example 5: 1H-NMR (400.0 MHz, d6-DMSO):
δ = 9.127 (15.3); 9.121 (16.0); 9.017 (3.7); 9.001 (7.8); 8.986 (3.8); 8.484 (14.4); 8.479 (14.6); 8.316 (0.7); 7.774
(7.5); 7.754 (12.4); 7.732 (8.7); 7.714 (6.0); 7.665 (6.1); 7.646 (7.8); 7.627 (2.9); 7.460 (9.0); 7.441 (8.0); 4.323
(3.3); 4.308 (3.5); 4.287 (7.6); 4.271 (7.4); 4.251 (3.9); 4.235 (3.6); 3.419 (0.4); 3.377 (56.4); 3.329 (346.1); 2.676
(1.3); 2.671 (1.8); 2.667 (1.4); 2.524 (4.6); 2.511 (100.7); 2.507 (201.5); 2.502 (267.0); 2.498 (201.7); 2.333 (1.3);
2.329 (1.8); 2.325 (1.3); 2.075 (1.9); 1.989 (1.0); 1.234 (0.6); 1.175 (0.6); 0.146 (1.9); 0.008 (16.2); 0.000 (410.0); −0.009
(21.5); −0.150 (1.9)
Example 6: 1H-NMR (400.0 MHz, d6-DMSO):
δ = 9.120 (15.4); 9.114 (16.0); 9.005 (3.6); 8.990 (7.5); 8.974 (3.6); 8.947 (12.4); 8.936 (12.5); 8.532 (14.9); 8.526
(15.1); 8.317 (0.7); 8.058 (11.9); 8.047 (12.4); 7.778 (7.2); 7.758 (10.1); 7.750 (3.9); 7.731 (8.2); 7.712 (5.8); 7.667
(5.8); 7.648 (7.4); 7.629 (2.7); 7.469 (8.7); 7.450 (7.6); 5.757 (3.1); 4.324 (3.3); 4.309 (3.4); 4.288 (7.6); 4.272 (7.3);
4.251 (3.8); 4.236 (3.6); 3.330 (319.5); 2.676 (1.5); 2.672 (2.0); 2.667 (1.5); 2.663 (0.8); 2.525 (5.0); 2.511 (110.7);
2.507 (225.7); 2.503 (299.6); 2.498 (223.2); 2.494 (113.2); 2.334 (1.4); 2.329 (2.0); 2.325 (1.5); 1.233 (0.5); 0.146
(0.8); 0.008 (5.9); 0.000 (177.3); −0.008 (7.9); −0.150 (0.8)
Example 7: ¹H-NMR(400.0 MHz, d₆-DMSO):
δ = 9.255(4.0); 9.240(7.9); 9.224(4.1); 9.111(15.6); 9.105(16.0); 9.023(12.6); 9.018(15.2); 8.970(15.8); 8.964(13.8); 8.953
(12.8); 8.942(12.2); 8.875(0.4); 8.871(0.4); 8.797(0.7); 8.793(0.7); 8.590(0.7); 8.585(0.7); 8.555(15.6); 8.550(15.5); 8.317(0.6);
8.296(0.4); 8.066(12.0); 8.056(12.2); 7.716(0.5); 7.695(0.5); 4.415(3.6); 4.399(3.7); 4.378(8.3); 4.362(8.0); 4.340(4.3);
4.324(4.0); 4.220(0.4); 4.204(0.4); 3.330(152.0); 2.677(1.3); 2.672(1.7); 2.668(1.3); 2.512(94.5); 2.508(184.8); 2.503(241.4);
2.499(181.6); 2.334(1.1); 2.330(1.6); 2.326(1.2); 1.990(0.6); 1.259(0.4); 1.233(0.5); 1.175(0.4); 0.008(0.8); 0.000(17.9)
Example 8: ¹H-NMR(400.0 MHz, d₆-DMSO):
δ = 9.277(2.6); 9.262(5.4); 9.246(2.6); 9.192(10.3); 9.187(10.6); 9.028(8.2); 9.022(10.2); 8.981(7.4); 8.974(16.0); 8.968(9.4);
8.703(10.4); 8.697(10.4); 8.318(0.4); 7.212(8.9); 7.206(9.0); 4.430(2.4); 4.414(2.4); 4.393(5.5); 4.377(5.2); 4.355(2.9); 4.339
(2.6); 4.038(0.8); 4.021(0.8); 3.331(103.2); 2.677(0.8); 2.673(1.1); 2.668(0.9); 2.564(0.5); 2.526(3.2); 2.512(59.8); 2.508
(119.5); 2.504(156.9); 2.499(115.6); 2.335(0.7); 2.330(0.9); 2.326(0.7); 1.990(3.4); 1.259(0.3); 1.234(0.5); 1.194(0.9); 1.176
(1.8); 1.158(0.9); 0.008(0.4); 0.000(12.0); −0.008(0.4)
Example 9: ¹H-NMR(400.0 MHz, d₆-DMSO):
δ = 9.173(3.5); 9.157(7.7); 9.142(4.0); 9.123(15.6); 9.117(16.0); 8.949(12.1); 8.938(12.2); 8.804(9.7); 8.795(8.3); 8.793(8.5);
8.580(1.4); 8.575(1.4); 8.544(15.0); 8.539(15.0); 8.318(0.4); 8.062(11.7); 8.052(12.1); 7.936(6.7); 7.919(8.9); 7.917(8.8);
7.904(1.0); 7.803(7.9); 7.791(7.8); 7.783(6.4); 7.772(6.1); 4.360(3.2); 4.345(3.3); 4.324(7.6); 4.308(7.4); 4.287(4.0); 4.271
(3.8); 4.242(0.5); 4.227(0.4); 3.333(231.3); 2.677(0.8); 2.673(1.2); 2.668(0.9); 2.526(3.0); 2.521(4.6); 2.512(62.1); 2.508
(128.2); 2.504(170.7); 2.499(126.7); 2.495(63.9); 2.335(0.8); 2.330(1.2); 2.326(0.9); 1.990(1.0); 1.233(0.5); 1.176(0.6); 0.146
(0.3); 0.008(2.7); 0.000(84.0); −0.008(3.4); −0.150(0.4)
Example 10: ¹H-NMR(400.0 MHz, d₆-DMSO):
δ = 9.444(12.7); 9.287(2.7); 9.272(5.9); 9.256(3.0); 9.203(11.4); 9.197(11.7); 9.023(9.0); 9.017(11.1); 8.971(11.2); 8.965(9.4);
8.712(11.5); 8.707(11.4); 8.405(16.0); 8.313(1.8); 8.141(11.1); 4.428(2.7); 4.412(2.7); 4.390(6.1); 4.374(5.9); 4.352(3.2);
4.336(2.9); 3.738(0.4); 3.355(2895.7); 2.677(4.5); 2.673(5.8); 2.669(4.4); 2.508(673.1); 2.504(853.5); 2.500(627.8); 2.335
(4.2); 2.331(5.6); 2.326(4.2); 2.288(0.6); 2.074(1.1); 1.910(4.6); 0.000(55.4)
Example 11: ¹H-NMR(400.0 MHz, d₆-DMSO):
δ = 9.200(15.5); 9.194(16.0); 9.024(3.7); 9.008(7.9); 8.993(3.9); 8.974(10.7); 8.969(10.9); 8.681(15.3); 8.676(15.1); 8.317
(0.6); 7.780(7.6); 7.760(10.8); 7.734(8.6); 7.716(6.0); 7.669(6.1); 7.650(7.8); 7.631(2.9); 7.476(9.1); 7.457(8.0); 7.205(13.2);
7.199(13.2); 4.340(3.4); 4.325(3.5); 4.304(7.7); 4.288(7.4); 4.267(3.9); 4.252(3.6); 3.336(388.4); 2.677(1.4); 2.672(1.9); 2.668
(1.4); 2.525(4.9); 2.508(218.6); 2.503(286.6); 2.499(213.2); 2.334(1.4); 2.330(1.9); 2.325(1.4); 2.076(6.5); 1.909(0.5);
0.008(0.6); 0.000(19.2); −0.008(0.8)
Example 12: ¹H-NMR(400.0 MHz, d₆-DMSO):
δ = 9.141(8.6); 9.136(8.9); 9.037(16.0); 9.011(2.2); 8.996(4.5); 8.981(2.2); 8.566(8.5); 8.561(8.6); 8.317(0.4); 8.071(14.7);
7.778(4.4); 7.759(6.1); 7.750(2.3); 7.731(5.0); 7.712(3.5); 7.667(3.6); 7.648(4.5); 7.629(1.6); 7.469(5.2); 7.451(4.6); 4.327
(2.0); 4.311(2.1); 4.290(4.5); 4.275(4.3); 4.253(2.3); 4.238(2.1); 3.338(195.8); 2.676(1.0); 2.672(1.4); 2.668(1.1); 2.507(158.5);
2.503(205.0); 2.499(155.4); 2.334(1.0); 2.330(1.4); 2.325(1.0); 2.076(0.9); 0.000(11.5)
Example 13: ¹H-NMR(400.0 MHz, d₆-DMSO):
δ = 9.466(0.5); 9.461(0.5); 9.202(15.2); 9.197(16.0); 9.189(4.2); 9.173(7.7); 9.158(3.6); 9.089(0.4); 9.021(0.5); 8.976(10.2);
8.972(10.4); 8.805(8.0); 8.793(8.1); 8.709(0.8); 8.692(14.8); 8.687(14.5); 8.407(0.4); 8.317(1.4); 7.980(0.4); 7.957(0.5); 7.940
(6.8); 7.921(8.8); 7.804(7.3); 7.792(7.2); 7.784(6.0); 7.773(5.6); 7.228(0.7); 7.220(0.8); 7.210(12.9); 7.203(12.8); 7.093
(0.5); 7.087(0.5); 4.374(3.3); 4.358(3.5); 4.337(7.6); 4.321(7.3); 4.300(4.1); 4.284(3.5); 3.568(0.6); 3.392(0.6); 3.332(1125.8);
3.276(0.6); 2.891(0.7); 2.731(0.7); 2.676(3.5); 2.671(4.8); 2.667(3.7); 2.565(0.6); 2.560(0.6); 2.524(12.4); 2.507(555.7);
2.502(731.0); 2.498(546.9); 2.333(3.5); 2.329(4.8); 2.325(3.7); 2.075(3.9); 1.235(0.3); 0.008(1.3); 0.000(42.7); −0.008(1.9)
Example 14: ¹H-NMR(400.0 MHz, d₆-DMSO):
δ = 9.466(3.3); 9.460(2.7); 9.443(12.6); 9.216(11.5); 9.211(11.9); 9.195(2.9); 9.181(5.7); 9.165(2.7); 9.131(0.7); 9.116(1.4);
9.100(0.7); 9.070(1.1); 8.857(2.8); 8.804(7.6); 8.795(7.0); 8.728(2.6); 8.722(2.6); 8.702(11.2); 8.697(11.2); 8.614(0.9); 8.609
(0.8); 8.576(0.4); 8.433(3.7); 8.404(16.0); 8.317(5.7); 8.265(1.1); 7.985(1.3); 7.967(1.6); 7.951(0.7); 7.937(5.2); 7.919(6.7);
7.803(5.9); 7.791(5.9); 7.784(5.4); 7.771(4.5); 4.372(2.9); 4.356(3.1); 4.335(6.2); 4.319(6.3); 4.298(3.4); 4.283(3.0); 4.259
(0.5); 4.249(0.5); 4.224(0.3); 3.428(0.3); 3.376(0.7); 3.329(824.8); 3.275(0.3); 2.676(5.5); 2.671(7.6); 2.667(5.7); 2.638
(0.4); 2.619(0.3); 2.524(18.7); 2.507(858.7); 2.502(1129.1); 2.498(837.7); 2.333(5.3); 2.329(7.4); 2.324(5.5); 2.075(5.7); 1.990
(0.4); 1.233(0.4); 0.146(0.4); 0.008(2.0); 0.000(66.1); −0.150(0.3)
Example 15: ¹H-NMR(400.0 MHz, d₆-DMSO):
δ = 8.965(0.7); 8.950(0.4); 8.939(1.3); 8.934(1.3); 8.613(1.9); 8.430(1.3); 8.425(1.3); 8.322(2.1); 7.780(0.6); 7.761(0.9); 7.729
(0.7); 7.710(0.5); 7.668(0.5); 7.649(0.6); 7.476(0.7); 7.457(0.6); 5.237(1.0); 5.215(1.1); 5.192(0.4); 4.287(0.6); 4.271(0.6);
3.934(3.0); 3.329(14.2); 2.525(0.5); 2.507(20.4); 2.503(26.0); 2.498(19.1); 1.909(0.7); 1.069(16.0); 0.008(0.5); 0.000(12.8);
−0.009(0.6)
Example 16: ¹H-NMR(400.0 MHz, d₆-DMSO):
δ = 9.151(2.3); 9.136(5.0); 9.120(2.4); 8.941(9.3); 8.936(9.8); 8.804(4.9); 8.794(4.7); 8.792(4.9); 8.615(14.3); 8.441(9.7); 8.437
(9.8); 8.324(16.0); 7.939(4.2); 7.922(5.2); 7.920(5.3); 7.889(0.6); 7.800(4.6); 7.788(4.5); 7.780(3.8); 7.768(3.6); 5.263
(2.4); 5.241(7.7); 5.218(8.2); 5.195(2.8); 4.357(2.0); 4.341(2.0); 4.319(4.6); 4.304(4.4); 4.282(2.3); 4.266(2.1); 4.056(0.9); 4.038
(2.7); 4.020(2.8); 4.002(0.9); 3.330(204.4); 2.891(0.6); 2.732(0.5); 2.676(0.9); 2.672(1.2); 2.668(0.9); 2.525(3.2); 2.521
(4.8); 2.512(64.8); 2.507(134.7); 2.503(180.9); 2.498(136.4); 2.494(70.2); 2.334(0.9); 2.330(1.2); 2.325(0.9); 1.989(12.0);
1.910(0.4); 1.259(0.6); 1.235(0.4); 1.193(3.3); 1.175(6.5); 1.158(3.2); 0.146(0.4); 0.008(3.0); 0.000(97.0); −0.009(4.4); −0.150
(0.4)
Example 17: ¹H-NMR(400.0 MHz, d₆-DMSO):
δ = 9.220(2.5); 9.204(5.2); 9.189(2.5); 9.022(7.6); 9.016(9.3); 8.968(9.4); 8.963(8.0); 8.936(9.3); 8.932(9.7); 8.622(14.4); 8.456
(9.9); 8.452(9.9); 8.329(16.0); 8.317(0.8); 5.757(3.7); 5.265(2.5); 5.243(8.0); 5.220(8.4); 5.197(2.9); 4.410(2.2); 4.394
(2.2); 4.373(4.9); 4.357(4.8); 4.335(2.5); 4.319(2.4); 4.056(1.2); 4.038(3.7); 4.020(3.7); 4.002(1.3); 3.934(0.7); 3.569(2.0); 3.328
(100.7); 2.676(1.0); 2.672(1.4); 2.667(1.1); 2.507(161.6); 2.503(212.6); 2.498(164.1); 2.334(1.0); 2.329(1.4); 2.325(1.1);
1.989(15.4); 1.812(0.5); 1.397(0.5); 1.298(0.4); 1.259(1.8); 1.250(0.7); 1.235(2.7); 1.193(4.2); 1.175(8.2); 1.158(4.6); 1.117
(0.6); 1.069(4.3); 0.854(0.4); 0.146(0.4); 0.008(3.3); 0.000(89.0); −0.150(0.4)
Example 18: ¹H-NMR(400.0 MHz, d₆-DMSO):
δ = 9.166(2.2); 9.151(4.8); 9.142(9.9); 9.137(11.0); 9.037(16.0); 8.803(4.7); 8.791(4.8); 8.575(8.7); 8.570(8.7); 8.314(0.4);
8.071(14.7); 7.934(4.1); 7.915(5.2); 7.800(4.2); 7.788(4.2); 7.780(3.5); 7.768(3.3); 4.361(2.0); 4.346(2.1); 4.324(4.6); 4.309
(4.4); 4.287(2.3); 4.272(2.2); 3.318(72.2); 2.675(0.8); 2.671(1.1); 2.667(0.9); 2.541(1.8); 2.524(3.0); 2.507(129.3); 2.502
(169.4); 2.498(126.8); 2.333(0.8); 2.329(1.1); 2.325(0.8); 0.146(0.8); 0.008(6.4); 0.000(166.7); −0.008(8.1); −0.150(0.8)

### FORMULATION EXAMPLES

An example for a formulation according to the present invention is the following:
8 mg compound of Example 3
0.2 mL Diethylene glycol monoethyl ether
0.2 mL Polyoxyl 35 Castor Oil
1.6 mL physiological sodium chloride solution

An example for a preparation of such a formulation is as follows. The compound of the present invention was dissolved in 1 part diethylene glycol monoethyl ether and mixed with 1 part Polyoxyl 35 Castor Oil and 8 parts physiological sodium chloride solution.

Such a formulation is suitable for oral or parenteral application.

Formulations of other compounds of the present invention can be prepared in an analogue way and show analogue or identical compositions.

### BIOLOGICAL EXAMPLES

### Example A: In Vitro Efficacy Test

In Vitro Assay in Cooperia curticei

To produce a suitable preparation of active compound, 10 mg of active compound are dissolved in 0.5 ml dimethyl sulfoxide, and the concentrate is diluted with “Ringer's solution” to the desired concentration.

Approximately 40 nematode larvae (Cooperia curticei) are transferred into a test tube containing the compound solution. After 5 days percentage of larval mortality is recorded. 100% efficacy means all larvae are killed; 0% efficacy means no larvae are killed.

In this test for example, the following compounds from the preparation examples showed good activity of 100% at an application rate of 20 ppm: 1, 2, 3, 5, 6, 8, 9, 10, 11, 12, 13, 14, 15, 16, 17, 18.

In this test for example, the following compounds from the preparation examples showed good activity of at least 90% at an application rate of 4 ppm: 1, 2, 3, 4, 5, 6, 8, 9, 10, 11, 12, 13, 14, 15, 18.

### Example B: In Vitro Efficacy Test

In Vitro Assay in Haemonchus contortus

To produce a suitable preparation of active compound, 10 mg of active compound are dissolved in 0.5 ml dimethyl sulfoxide, and the concentrate is diluted with “Ringer's solution” to the desired concentration.

Approximately 40 larvae of the red stomach worm (Haemonchus contortus) are transferred into a test tube containing compound solution. After 5 days percentage of larval mortality are recorded. 100% efficacy means all larvae are killed, 0% efficacy means no larvae are killed.

In this test for example, the following compounds from the preparation examples showed good activity of 100% at an application rate of 20 ppm: 3, 6, 10, 11, 12, 14, 18.

In this test for example, the following compounds from the preparation examples showed good activity of 90% at an application rate of 20 ppm: 2 and 5

### Example C: In Vitro Efficacy Test

In Vitro Assay in Nippostrongylus Brasiliensis

Adult Nippostrongylus brasiliensis washed with saline buffer containing 100 U/ml penicillin, 0.1 mg/ml streptomycin and 2.5 μg/ml amphotericin B. Test compounds were dissolved in DMSO, and worms were incubated in medium in a final concentration of 10 μg/ml. An aliquot of the medium was used to determine the acetylcholine esterase activity in comparison to a negative control. The principle of measuring acetylcholine esterase as readout for anthelmintic activity was described in Rapson et al (1986) and Rapson et al (1987).

For the following examples, activity (reduction of AChE compared to negative control) was 75% or higher at 10 μg/ml: 1, 3, 6, 8, 11, 12

### Example D: In Vivo Efficacy Test

Haemonchus contortus/Trichostrongylus colubriformis/gerbil

Gerbils, experimentally infected with Haemonchus and/or Trichostrongylus, were treated once during late prepatency. Test compounds were formulated as solutions or suspensions and applied intraperitoneally or orally.

Efficacy was determined per group as reduction of worm count in stomach and small intestine, respectively, after necropsy compared to worm count in an infected and placebo-treated control group.

The following examples were tested and had an activity of 85% or higher at the given treatment:

| Treatment | Haemonchus | Trichostrongylus |
|---|---|---|
| 20 mg/kg intraperitoneally | 3, 12 | 3, 12 |
| 20 mg/kg subcutaneously | 6 | 6 |
| 10 mg/kg subcutaneously | 3 | 3 |
| 5 mg/kg subcutaneously | 12 | 12 |

1: A compound of formula (I) 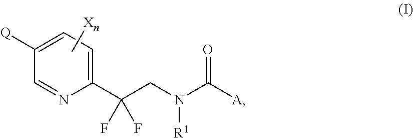
wherein
R¹ is selected from the group consisting of hydrogen, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl, —S(O)₂—C₁-C₄-alkyl, and —S(O)₂—C₁-C₄-halogenoalkyl having 1 to 5 halogen atoms,
n is 0, 1, 2 or 3,
each X is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino,
Q represents an aromatic 5-membered heterocyclic ring containing one to four heterotaoms chosen from N, S and O and bearing the substituent(s) Yₘ, with
m is 0, 1, 2, 3 or 4, limited by the number of available positions in Q to which a substituent Y can be connected, and
each Y is independently selected from the group consisting of hydrogen, oxo, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, —CH₂—S—C₁-C₈-alkyl, —CH₂—S(O)—C₁-C₈-alkyl, —CH₂—S(O)₂—C₁-C₈-alkyl, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S— phenyl and phenylamino, and
A represents a phenyl group of the formula (A1) 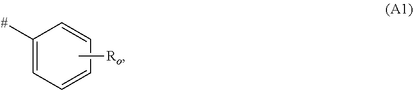
in which
# depicts the bond which connects A to the rest of the molecule,
o is 0, 1, 2, 3, 4 or 5, and
each R is independently selected from the group consisting of halogen, nitro, —OH, NH₂, SH, SF₅, CHO, OCHO, NHCHO, COOH, cyano, C₁-C₈-alkyl, C₁-C₈-halogenoalkyl having 1 to 9 halogen atoms, C₂-C₈-alkenyl, C₂-C₈-alkynyl, C₃-C₆-cycloalkyl, —S—C₁-C₈-alkyl, —S—C₁-C₈-halogenoalkyl having 1 to 5 halogen atoms, C₁-C₈-alkoxy, C₁-C₈-halogenoalkoxy having 1 to 5 halogen atoms, C₁-C₈-alkoxy-C₂-C₈-alkenyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, C₁-C₈-alkylsulfonamide, —NH(C₁-C₈-alkyl), N(C₁-C₈-alkyl)₂, phenyl (optionally substituted by C₁-C₆-alkoxy) and phenoxy, or two R bonded to adjacent carbon atoms together represent —O(CH₂)ₚO—, wherein p represents 1 or 2, or
A represents a heterocycle of the formula (Het-1) 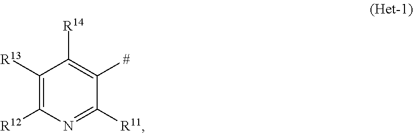
in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and —S-phenyl (optionally substituted by halogen or C₁-C₄-alkyl), and
R¹², R¹³ and R¹⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, or
A represents a heterocycle of the formula (Het-2) 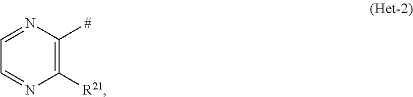
in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms,
or a pharmaceutically acceptable salt, N-oxide, metal complex or metalloid complex thereof,
with the proviso that if
A is 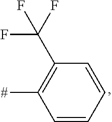
in which
# depicts the bond which connects A to the rest of the molecule,
R¹ is hydrogen,
X is chlorine at position 3 of the pyridine ring where it is connected to, and
n is 1, then
Q is not one of the following 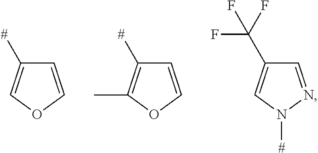
in which
# depicts the bond which connects Q to the rest of the molecule.

2: The compound according to claim 1, wherein
Q represents a 5-membered ring selected from the group consisting of Q-1 to Q-47: 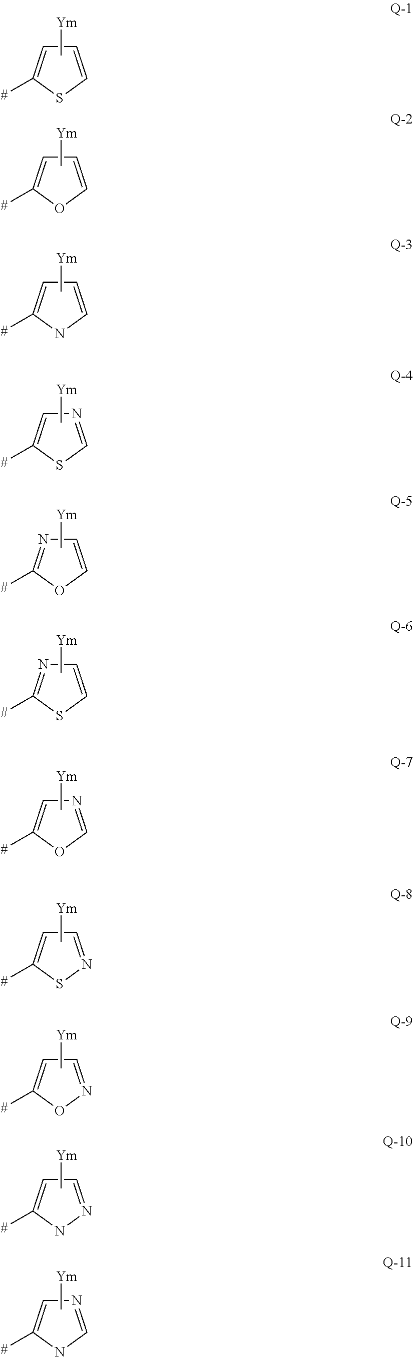 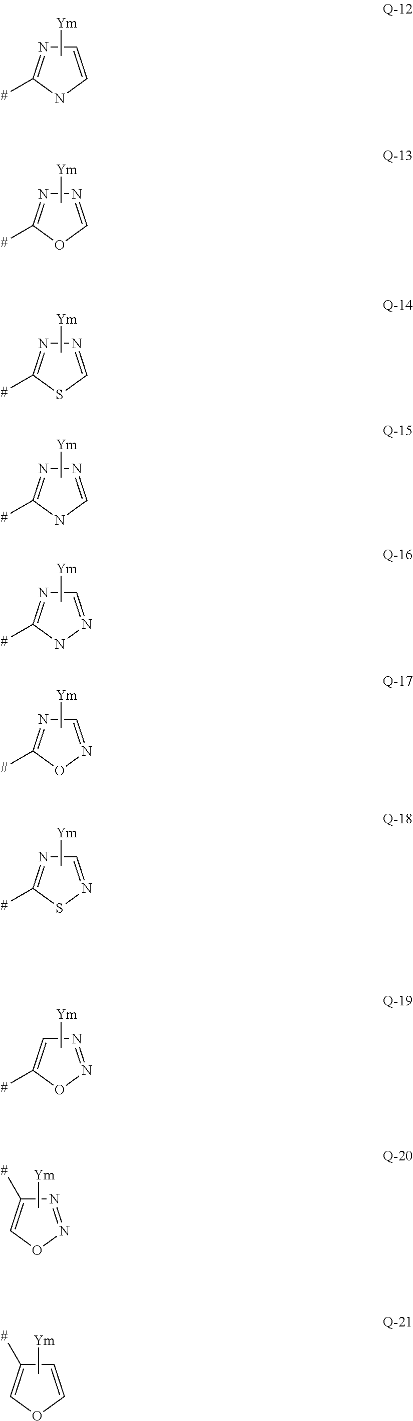 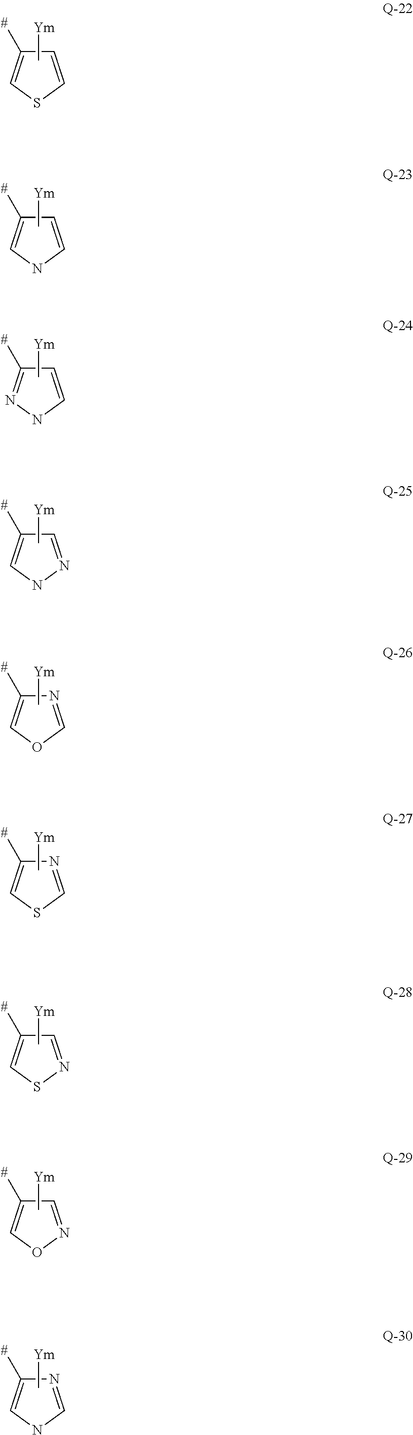 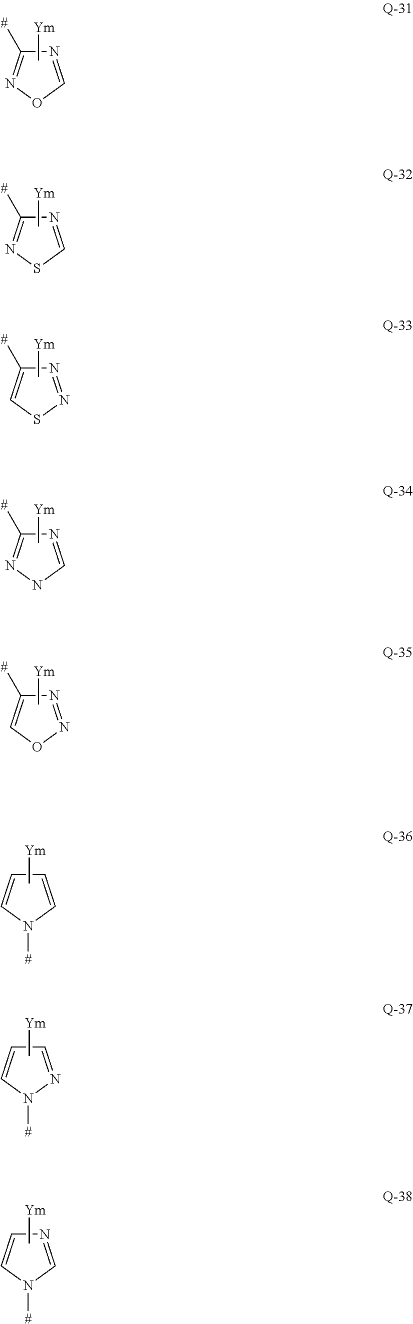 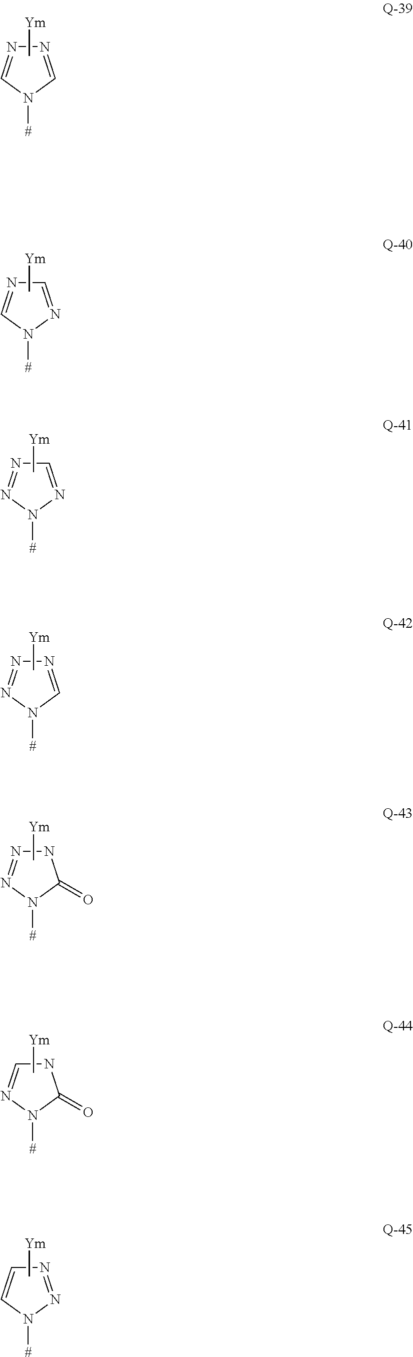 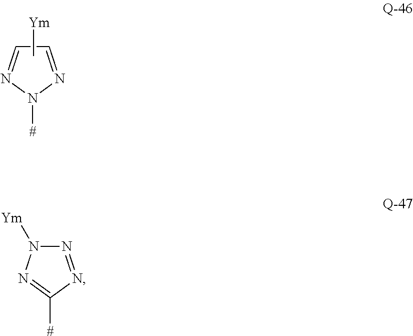
in which
# depicts the bond which connects Q to the rest of the molecule,
with m and Y having the meaning as described before.

3: The compound according to claim 2, wherein
n is 1,
X is selected from the group consisting of hydrogen, halogen, nitro, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
Q represents an optionally mono- or polysubstituted heteroaromatic ring from the group consisting of Q-1, Q-2, Q-3, Q-4, Q-5, Q-6, Q-7, Q-8, Q-9, Q-10, Q-11, Q-12, Q-13, Q-14, Q-15, Q-16, Q-18, Q-21, Q-22, Q-23, Q-24, Q-25, Q-26, Q-27, Q-28, Q-29, Q-30, Q-31, Q-32, Q-33, Q-34, Q-36, Q-37, Q-38, Q-39, Q-40, Q-41 and Q-44,
with
m is 0, 1 or 2, limited by the number of available positions in Q to which a substituent Y can be connected, and
each Y is independently selected from the group consisting of hydrogen, —CF₃, —CH₂CF₃, methyl, ethyl, fluorine, chlorine, bromine, iodine, cyano, —OCH₃, —OCH₂CH₃, —OCH(CH₃)₂, —OCH₂CF₃, —CH₂—S(O)₂—CH₃,
R¹ is selected from the group consisting of hydrogen, C₁-C₄-alkyl, C₁-C₄-alkoxy-C₁-C₄-alkyl, C₃-C₆-cycloalkyl, C₁-C₄-alkylcarbonyl, C₁-C₄-alkoxycarbonyl, and
A represents a phenyl group of formula (A1) 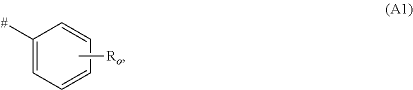
in which
# depicts the bond which connects A to the rest of the molecule,
o is 0, 1 or 2, and
each R is independently selected from the group consisting of halogen, nitro, —OH, cyano, C₁-C₄-alkyl, C₁-C₄-halogenoalkyl having 1 to 5 halogen atoms, C₃-C₆-cycloalkyl, C₁-C₄-alkoxy, C₁C₁-C₄-alkoxycarbonyl, —NH(C₁-C₄-alkyl), phenyl (optionally substituted by C₁-C₄-alkoxy) and phenoxy, or
A represents a heterocycle of the formula (Het-1) 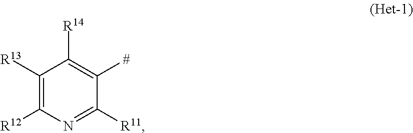
in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, —S(O)—C₁-C₄-alkyl, —S(O)₂₋C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and —S-phenyl (optionally substituted by halogen or C₁-C₄-alkyl), and
R¹², R¹³ and R¹⁴, which may the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, or
A represents a heterocycle of the formula (Het-2) 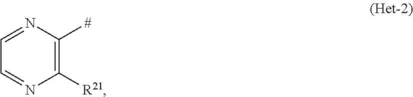
in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms.

4: The compound according to claim 2, wherein
n is 1,
X is selected from the group consisting of hydrogen, halogen, nitro, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
Q represents a 5-membered ring selected from the group consisting of Q-1, Q-4, Q-6, Q-10, Q-21, Q-23, Q-24, Q-25, Q-27, Q-37, Q-41 and Q-44,
with
m is 0, 1 or 2, limited by the number of available positions in Q to which a substituent Y can be connected, and
each Y is independently selected from the group consisting of hydrogen, —CF₃, —CH₂CF₃, methyl, ethyl, fluorine, chlorine, bromine, iodine, cyano, —OCH₃, —OCH₂CH₃,
R¹ is hydrogen, and
A represents a phenyl group of formula (A1) 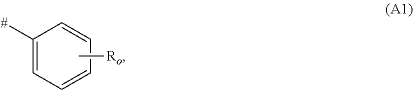
in which
# depicts the bond which connects A to the rest of the molecule,
o is 1 or 2, and
each R is independently selected from the group consisting of halogen, nitro, —OH, cyano, C₁-C₄-alkyl, C₁-C₄-halogenoalkyl having 1 to 5 halogen atoms, C₃-C₆-cycloalkyl, C₁-C₄-alkoxy, or
A represents a heterocycle of the formula (Het-1) 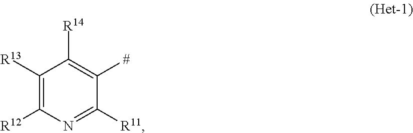
in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, C₁-C₄-halogenoalkoxy having 1 to 5 halogen atoms, and
R¹², R¹³ and R¹⁴, which may the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, or
A represents a heterocycle of the formula (Het-2) 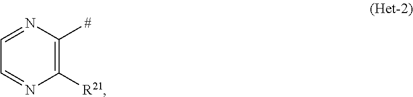
in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms.

5: The compound according to claim 2, wherein
n is 1,
X is selected from the group consisting of hydrogen, halogen, —CF₃,
Q represents a 5-membered ring selected from the group consisting of Q-21, Q-23, Q-25, Q-37 and Q-44,
with
m is 0, 1 or 2, limited by the number of available positions in Q to which a substituent Y can be connected, and
each Y is independently selected from the group consisting of hydrogen, —CF₃, —CH₂CF₃, methyl, ethyl, fluorine, chlorine,
R¹ is hydrogen, and
A represents a phenyl group of formula (A1) 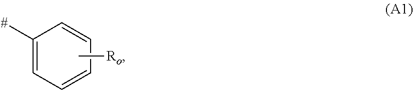
in which
# depicts the bond which connects A to the rest of the molecule,
o is 1 or 2, and
each R is independently selected from the group consisting of halogen, nitro, —OH, cyano, methyl and —CF₃, or
A represents a heterocycle of the formula (Het-1) 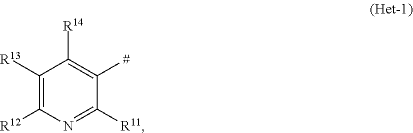
in which
# depicts the bond which connects A to the rest of the molecule,
R¹¹ is selected from the group consisting of hydrogen, halogen, methyl and —CF₃, and
R¹², R¹³ and R¹⁴, which may the same or different, are selected from the group consisting of hydrogen, halogen and —CF₃, or
A represents a heterocycle of the formula (Het-2) 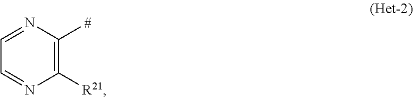
in which
# depicts the bond which connects A to the rest of the molecule, and
R²¹ is selected from the group consisting of hydrogen, halogen, methyl and —CF₃.

6: The compound according to claim 1, wherein
n is 1,
X is selected from the group consisting of hydrogen, halogen, nitro, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
Q is selected from the group consisting of 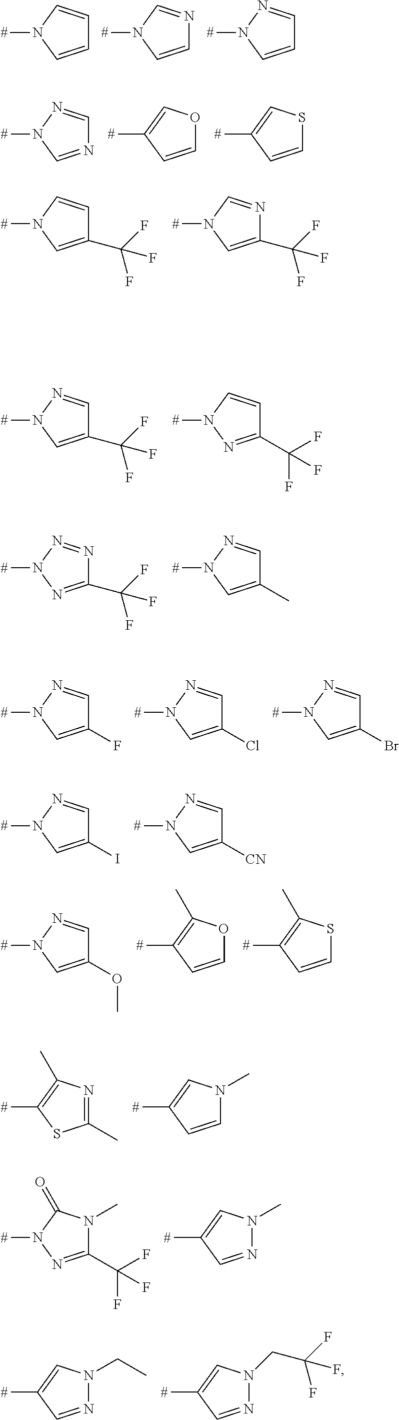
in which
# depicts the bond which connects Q to the rest of the molecule,
R¹ is hydrogen, and
A is selected from the group consisting of 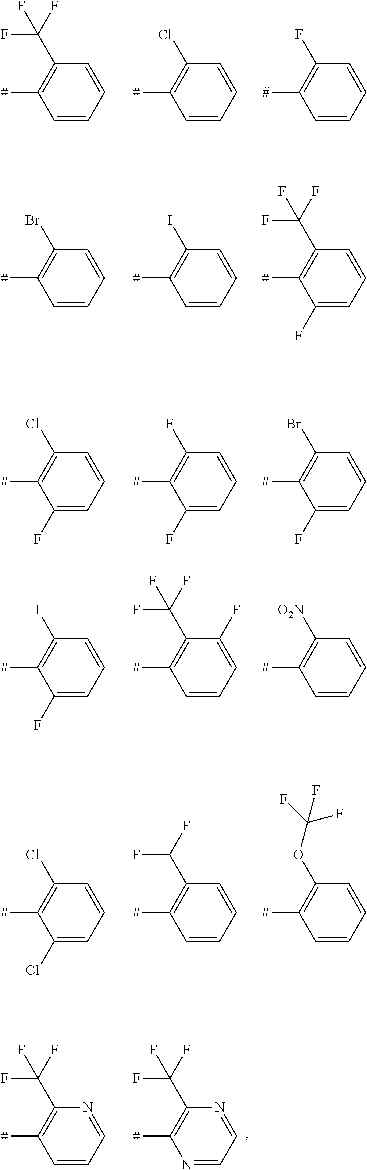
in which
# depicts the bond which connects A to the rest of the molecule.

7: The compound according to claim 1, wherein
n is 1,
X is chlorine,
Q is selected from the group consisting of 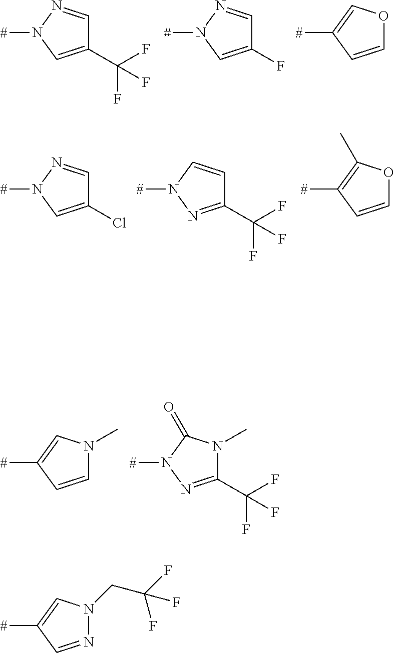
in which
# depicts the bond which connects Q to the rest of the molecule,
R¹ is hydrogen, and
A is selected from the group consisting of 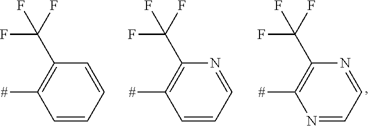
in which
# depicts the bond which connects A to the rest of the molecule.

8: A compound of formula (I-1) 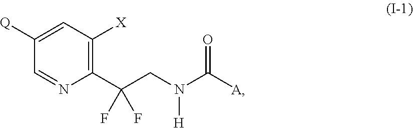
wherein
Q is a 5-membered ring selected from the group consisting of Q-1, Q-4, Q-6, Q-10, Q-21, Q-23, Q-24, Q-25, Q-27, Q-37, Q-41 and Q-44, with
m is 0, 1 or 2, limited by the number of available positions in Q to which a substituent Y can be connected, and
each Y is independently selected from the group consisting of hydrogen, halogen, C₁-C₄-alkyl and C₁-C₄-halogenoalkyl having 1 to 5 halogen atoms,
X is selected from the group consisting of fluorine, chlorine and trifluoro methyl, and
A represents a phenyl group of formula (A1) 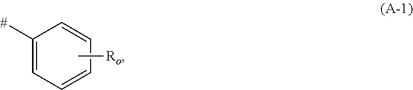
in which
# depicts the bond which connects A to the rest of the molecule,
o is 0, 1 or 2, and
each R is independently selected from the group consisting of halogen, nitro, —OH, cyano, C₁-C₄-alkyl, C₁-C₄-halogenoalkyl having 1 to 5 halogen atoms, C₃-C₆-cycloalkyl, C₁-C₄-alkoxy, C₁C₁-C₄-alkoxycarbonyl, —NH(C₁-C₄-alkyl), phenyl (optionally substituted by C₁-C₄-alkoxy) and phenoxy, or
A represents a heterocycle of the formula (Het-1) 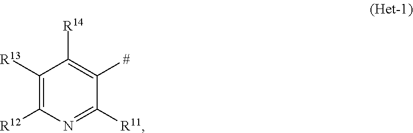
in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, —S(O)—C₁-C₄-alkyl, —S(O)₂₋C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and —S-phenyl (optionally substituted by halogen or C₁-C₄-alkyl), and
R¹², R¹³ and R¹⁴, which may the same or different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, or
A represents a heterocycle of the formula (Het-2) 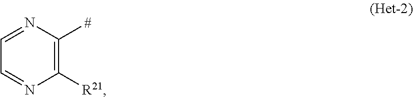
in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms,
with the proviso that if
A is 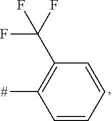
in which
# depicts the bond which connects A to the rest of the molecule, and
X is chlorine, then
Q is not one of the following 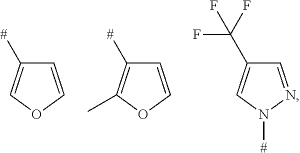
in which
# depicts the bond which connects Q to the rest of the molecule.

9: A compound of formula (I) 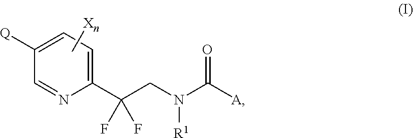
wherein
R¹ is selected from the group consisting of hydrogen, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl, —S(O)₂—C₁-C₄-alkyl, and —S(O)₂—C₁-C₄-halogenoalkyl having 1 to 5 halogen atoms,
n is 0, 1, 2 or 3,
each X is independently selected from the group consisting of hydrogen, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S-phenyl and phenylamino,
Q represents an aromatic 5-membered heterocyclic ring containing one to four heterotaoms chosen from N, S and O and bearing the substituent(s) Yₘ, with
m is 0, 1, 2, 3 or 4, limited by the number of available positions in Q to which a substituent Y can be connected, and
each Y is independently selected from the group consisting of hydrogen, oxo, halogen, nitro, cyano, hydroxy, amino, —SH, —SF₅, —CHO, —OCHO, —NHCHO, —COOH, —CONH₂, —CONH(OH), —OCONH₂, (hydroxyimino)-C₁-C₆-alkyl, C₁-C₈-alkyl, C₁-C₈-halogenoalkyl having 1 to 5 halogen atoms, C₂-C₈-alkenyl, C₂-C₈-alkynyl, C₁-C₈-alkylamino, di-(C₁-C₈-alkyl)amino, C₁-C₈-alkoxy, C₁-C₈-halogenoalkoxy having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₃-C₈-cycloalkyl, C₃-C₈-halogenocycloalkyl having 1 to 5 halogen atoms, C₁-C₈-alkylcarbonyl, C₁-C₈-halogenoalkylcarbonyl having 1 to 5 halogen atoms, —CONH(C₁-C₈-alkyl), —CON(C₁-C₈-alkyl)₂, —CONH(OC₁-C₈-alkyl), —CON(OC₁-C₈-alkyl)(C₁-C₈-alkyl), C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, —OCONH(C₁-C₈-alkyl), —OCON(C₁-C₈-alkyl)₂, —OCONH(OC₁-C₈-alkyl), —OCO(OC₁-C₈-alkyl), —S—C₁-C₈-alkyl, —S—C₁-C₈-halogenoalkyl having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, —CH₂—S—C₁-C₈-alkyl, —CH₂—S(O)—C₁-C₈-alkyl, —CH₂—S(O)₂—C₁-C₈-alkyl, (C₁-C₆-alkoxyimino)-C₁-C₆-alkyl, (C₂-C₆-alkenyloxyimino)-C₁-C₆-alkyl, (C₃-C₆-alkynyloxyimino)-C₁-C₆-alkyl, (benzyloxyimino)-C₁-C₆-alkyl, benzyloxy, —S-benzyl, benzylamino, phenoxy, —S— phenyl and phenylamino,
A represents a phenyl group of the formula (A1) 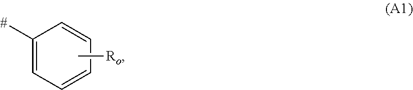
in which
# depicts the bond which connects A to the rest of the molecule,
o is 0, 1, 2, 3, 4 or 5, and
each R is independently selected from the group consisting of halogen, nitro, —OH, NH₂, SH, SF₅, CHO, OCHO, NHCHO, COOH, cyano, C₁-C₈-alkyl, C₁-C₈-halogenoalkyl having 1 to 9 halogen atoms, C₂-C₈-alkenyl, C₂-C₈-alkynyl, C₃-C₆-cycloalkyl, —S—C₁-C₈-alkyl, —S—C₁-C₈-halogenoalkyl having 1 to 5 halogen atoms, C₁-C₈-alkoxy, C₁-C₈-halogenoalkoxy having 1 to 5 halogen atoms, C₁-C₈-alkoxy-C₂-C₈-alkenyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, —S(O)—C₁-C₈-alkyl, —S(O)—C₁-C₈-halogenoalkyl having 1 to 5 halogen atoms, —S(O)₂—C₁-C₈-alkyl, —S(O)₂—C₁-C₈-halogenoalkyl having 1 to 5 halogen atoms, C₁-C₈-alkylsulfonamide, —NH(C₁-C₈-alkyl), N(C₁-C₈-alkyl)₂, phenyl (optionally substituted by C₁-C₆-alkoxy) and phenoxy, or two R bonded to adjacent carbon atoms together represent —O(CH₂)ₚO—, wherein p represents 1 or 2, or
A represents a heterocycle of the formula (Het-1) 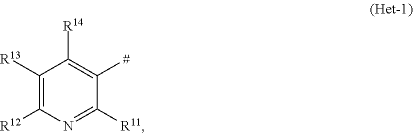
in which
# depicts the bond which connects A to the rest of the molecule,
R¹¹ is selected from the group consisting of hydrogen, halogen, hydroxy, cyano, C₁-C₄-alkyl, C₁-C₄-halogenoalkyl having 1 to 5 halogen atoms, C₁-C₄ alkoxy, —S—C₁-C₅-alkyl, S(O)—C₁-C₄-alkyl, —S(O)₂—C₁-C₄-alkyl, —S—C₂-C₅-alkenyl, —S—C₁-C₄-halogenoalkyl having 1 to 5 halogen atoms, C₁-C₄-halogenoalkoxy having 1 to 5 halogen atoms, phenyloxy (optionally substituted by halogen or C₁-C₄-alkyl) and —S-phenyl (optionally substituted by halogen or C₁-C₄-alkyl), and
R¹², R¹³ and R¹⁴, which may be the same or be different, are selected from the group consisting of hydrogen, halogen, cyano, C₁-C₄-alkyl, C₁-C₄-halogenoalkyl having 1 to 5 halogen atoms, C₁-C₄-alkoxy, —S—C₁-C₄-alkyl, C₁-C₄-halogenoalkoxy having 1 to 5 halogen atoms, —S(O)—C₁-C₄-alkyl, —S(O)₂—C₁-C₄-alkyl, or
A represents a heterocycle of the formula (Het-2) 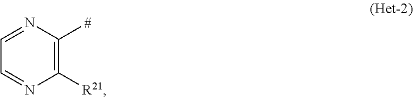
in which
# depicts the bond which connects A to the rest of the molecule, and
R²¹ is selected from the group consisting of hydrogen, halogen, C₁-C₄-alkyl and C₁-C₄-halogenoalkyl having 1 to 5 halogen atoms,
or a pharmaceutically acceptable salt, N-oxide, metal complex or metalloid complex thereof,
for use in the control, treatment and/or prevention of infections with helminths in animals and humans.

10: A pharmaceutical composition comprising at least one compound of formula (I) according to claim 1.

11-14. (canceled)

15: A method for the control, treatment and/or prevention of infections with helminths in animals and humans, comprising the step of administering an effective amount of a compound of formula (I) of claim 1 to an animal or human in need thereof.

16: A method for the control, treatment and/or prevention of infections with helminths in animals and humans, comprising the step of administering a pharmaceutical composition of claim 10 to an animal or human in need thereof.

17: A pharmaceutical composition comprising at least one compound of formula (I) according to claim 9.

18: A method for the control, treatment and/or prevention of infections with helminths in animals and humans, comprising the step of administering an effective amount of a compound of formula (I) of claim 9 to an animal or human in need thereof.

19: A method for the control, treatment and/or prevention of infections with helminths in animals and humans, comprising the step of administering a pharmaceutical composition of claim 17 to an animal or human in need thereof.
